(19) United States
(12) Patent Application Publication
(10) Pub. No.: US 2024/0173499 A1
(43) Pub. Date: May 30, 2024

(54) SYSTEMS AND METHODS FOR MANAGING BLOOD PRESSURE CONDITIONS OF A USER OF A RESPIRATORY THERAPY SYSTEM

(71) Applicant: ResMed Sensor Technologies Limited, Dublin 4 (IE)

(72) Inventors: Redmond SHOULDICE, Dublin (IE); Michael WREN, Dublin (IE)

(21) Appl. No.: 18/553,364

(22) PCT Filed: Mar. 29, 2022
(86) PCT No.: PCT/IB2022/052915

§ 371 Date: Sep. 29, 2023

Related U.S. Application Data
(60) Provisional application No. 63/169,147, filed on Mar. 31, 2021.

(51) Int. Cl. A61M 16/00; G16H 20/40; G16H 40/67

(52) U.S. Cl. CPC A61M 16/024; G16H 20/40; G16H 40/67; A61M 2230/30

(57) ABSTRACT

Various implementations of the present disclosure are directed to systems and methods for managing blood pressure conditions of a user of a respiratory therapy system by controlling operational parameters associated with the respiratory therapy system. The method includes receiving cardiovascular data associated with a user of a respiratory therapy system during a sleep session and determining, based at least in part on the received cardiovascular data, a value of a first physiological parameter associated with the user. The method further includes determining whether the value of the first physiological parameter satisfies a first condition and in response to the first physiological parameter satisfying the first condition, determining a modification of an operational parameter associated with the respiratory therapy system.

### CROSS REFERENCE TO RELATED APPLICATIONS

The present application claims the benefit of U.S. Provisional Patent Application No. 63/169,147 filed Mar. 31, 2021, and entitled “SYSTEMS AND METHODS FOR MANAGING BLOOD PRESSURE CONDITIONS OF A USER OF A RESPIRATORY THERAPY SYSTEM,” the disclosure of which is hereby incorporated by reference in its entirety.

### TECHNICAL FIELD

The present disclosure relates generally to systems and methods for managing physiological conditions of a user of a respiratory therapy system, and more particularly, to systems and methods for managing blood pressure conditions of a user of a respiratory therapy system by controlling operational parameters associated with the respiratory therapy system.

### BACKGROUND

Many individuals suffer from sleep-related and/or respiratory disorders such as, for example, Periodic Limb Movement Disorder (PLMD), Restless Leg Syndrome (RLS), Sleep-Disordered Breathing (SDB), apneas such as Obstructive Sleep Apnea (OSA), Central Sleep Apnea (CSA), mixed apneas and hypopneas, Respiratory Effort Related Arousal (RERA), Cheyne-Stokes Respiration (CSR), respiratory insufficiency, Obesity Hyperventilation Syndrome (OHS), Chronic Obstructive Pulmonary Disease (COPD), Neuromuscular Disease (NMD), and chest wall disorders. These disorders are often treated using a respiratory therapy system.

Users of the respiratory therapy system may have diagnosed or undiagnosed blood pressure conditions such as hypertension, e.g. nocturnal hypertension. It is generally understood that individuals with blood pressure conditions have a higher risk of heart failures and other cardiovascular diseases. Thus, users of the respiratory therapy system having continual or episodic blood pressure conditions at night or during a sleep session may be more susceptible to experiencing cardiovascular events, even if their blood pressure is otherwise normal or within an acceptable range during the daytime.

Current flow management algorithms for delivering pressurized air to the user of the respiratory therapy system attempt to deliver the pressurized air with a gradual progression of the air pressure in order to avoid discomfort of the user. The flow management algorithms may only increase air pressure when the number of respiratory events, such as residual Apnea-Hypopnea Index (AHI), exceeds a programmed threshold or severe respiratory events have happened. This, however, fails to account for clusters of apnea, hypopnea, subapnea events, or RERAs (in particular if measuring AHI without also measuring and considering Respiratory Disturbance Index (RDI)) that may not lead to the average residual AHI exceeding the programmed threshold, but nevertheless results in blood pressure events (e.g. random spikes, continually rising blood pressure for a period of time, etc.) as well as sympathetic (“fight or flight”) autonomous nervous system activations that make the user vulnerable to cardiovascular events. Accordingly, it is desirable to proactively or reactively reduce or avoid the occurrence of these blood pressure events and associated physiological events by controlling operational parameters associated with the respiratory therapy system, thereby effectively managing blood pressure conditions of the user.

### SUMMARY

According to some implementations of the present disclosure, a method includes receiving cardiovascular data associated with a user of a respiratory therapy system during a sleep session and determining, based at least in part on the received cardiovascular data, a value of a first physiological parameter associated with the user. The method further includes determining whether the value of the first physiological parameter satisfies a first condition and in response to the first physiological parameter satisfying the first condition, determining a modification of an operational parameter associated with the respiratory therapy system.

According to some implementations of the present disclosure, a system includes an electronic interface, a cardiovascular sensing mechanism communicatively coupled to the electronic interface, and a control system. The electronic interface is configured to receive cardiovascular data associated with a sleep session of a user. The control system includes one or more processors configured to execute machine-readable instructions to receive cardiovascular data associated with the user during the sleep session from the cardiovascular sensing mechanism. The control system is also configured to determine, based at least in part on the received cardiovascular data, a value of a first physiological parameter associated with the user. The control system is further configured to determine whether the value of the first physiological parameter satisfies a first condition. The control system is further configured to determine a modification of an operational parameter associated with the respiratory therapy system, in response to the value of the first physiological parameter satisfying the first condition.

The above summary is not intended to represent each implementation or every aspect of the present disclosure. Additional features and benefits of the present disclosure are apparent from the detailed description and figures set forth below.

### BRIEF DESCRIPTION OF THE DRAWINGS

FIG. 1 is a functional block diagram of a respiratory therapy system, according to some implementations of the present disclosure.

FIG. 2 is a perspective view of at least a portion of the system of FIG. 1, a user, and a bed partner, according to some implementations of the present disclosure.

FIG. 3 illustrates an exemplary timeline for a sleep session, according to some implementations of the present disclosure.

FIG. 4 illustrates an exemplary hypnogram associated with the sleep session of FIG. 3, according to some implementations of the present disclosure.

FIG. 5 illustrates a smart patch 600 which may be communicatively coupled to the respiratory therapy system and disposed on a skin of a user of the system, according to some implementations of the present disclosure.

FIG. 6 illustrates the smart patch 600 coupled to an inner surface of a user interface of the respiratory therapy system and contacting a skin of a user of the system, according to some implementations of the present disclosure.

FIG. 7 illustrates a flow diagram for a method for managing blood pressure conditions of a user of a respiratory therapy system, according to some implementations of the present disclosure.

FIG. 8 is a flowchart depicting a process for scoring sleep performance, according to certain aspects of the present disclosure.

While the present disclosure is susceptible to various modifications and alternative forms, specific implementations and embodiments thereof have been shown by way of example in the drawings and will herein be described in detail. It should be understood, however, that it is not intended to limit the present disclosure to the particular forms disclosed, but on the contrary, the present disclosure is to cover all modifications, equivalents, and alternatives falling within the spirit and scope of the present disclosure as defined by the appended claims.

### DETAILED DESCRIPTION

Many individuals suffer from sleep-related and/or respiratory disorders. Examples of sleep-related and/or respiratory disorders include Periodic Limb Movement Disorder (PLMD), Restless Leg Syndrome (RLS), Sleep-Disordered Breathing (SDB) such as Obstructive Sleep Apnea (OSA), Central Sleep Apnea (CSA) and other types of apneas such as mixed apneas and hypopneas, Respiratory Effort Related Arousal (RERA), Cheyne-Stokes Respiration (CSR), respiratory insufficiency, Obesity Hyperventilation Syndrome (OHS), Chronic Obstructive Pulmonary Disease (COPD), Neuromuscular Disease (NMD), and chest wall disorders.

Obstructive Sleep Apnea (OSA) is a form of Sleep Disordered Breathing (SDB), and is characterized by events including occlusion or obstruction of the upper air passage during sleep resulting from a combination of an abnormally small upper airway and the normal loss of muscle tone in the region of the tongue, soft palate and posterior oropharyngeal wall. More generally, an apnea generally refers to the cessation of breathing caused by blockage of the air (Obstructive Sleep Apnea) or the stopping of the breathing function (often referred to as Central Sleep Apnea). Typically, the individual will stop breathing for between about 15 seconds and about 30 seconds during an obstructive sleep apnea event.

Other types of apneas include hypopnea, hyperpnea, and hypercapnia. Hypopnea is generally characterized by slow or shallow breathing caused by a narrowed airway, as opposed to a blocked airway. Hyperpnea is generally characterized by an increase depth and/or rate of breathing. Hypercapnia is generally characterized by elevated or excessive carbon dioxide in the bloodstream, typically caused by inadequate respiration.

Cheyne-Stokes Respiration (CSR) is another form of sleep disordered breathing. CSR is a disorder of a patient's respiratory controller in which there are rhythmic alternating periods of waxing and waning ventilation known as CSR cycles. CSR is characterized by repetitive de-oxygenation and re-oxygenation of the arterial blood.

Obesity Hyperventilation Syndrome (OHS) is defined as the combination of severe obesity and awake chronic hypercapnia, in the absence of other known causes for hypoventilation. Symptoms include dyspnea, morning headache and excessive daytime sleepiness.

Chronic Obstructive Pulmonary Disease (COPD) encompasses any of a group of lower airway diseases that have certain characteristics in common, such as increased resistance to air movement, extended expiratory phase of respiration, and loss of the normal elasticity of the lung.

Neuromuscular Disease (NMD) encompasses many diseases and ailments that impair the functioning of the muscles either directly via intrinsic muscle pathology, or indirectly via nerve pathology. Chest wall disorders are a group of thoracic deformities that result in inefficient coupling between the respiratory muscles and the thoracic cage.

A Respiratory Effort Related Arousal (RERA) event is typically characterized by an increased respiratory effort for ten seconds or longer leading to arousal from sleep and which does not fulfill the criteria for an apnea or hypopnea event. RERAs are defined as a sequence of breaths characterized by increasing respiratory effort leading to an arousal from sleep, but which does not meet criteria for an apnea or hypopnea. These events must fulfil both of the following criteria: (1) a pattern of progressively more negative esophageal pressure, terminated by a sudden change in pressure to a less negative level and an arousal, and (2) the event lasts ten seconds or longer. In some implementations, a Nasal Cannula/Pressure Transducer System is adequate and reliable in the detection of RERAs. A RERA detector may be based on a real flow signal derived from a respiratory therapy device. For example, a flow limitation measure may be determined based on a flow signal. A measure of arousal may then be derived as a function of the flow limitation measure and a measure of sudden increase in ventilation. One such method is described in WO 2008/138040, assigned to ResMed Ltd., the disclosure of which is hereby incorporated by reference herein in its entirety.

These and other disorders are characterized by particular events (e.g., snoring, an apnea, a hypopnea, a restless leg, a sleeping disorder, choking, an increased heart rate, labored breathing, an asthma attack, an epileptic episode, a seizure, or any combination thereof) that occur when the individual is sleeping.

The Apnea-Hypopnea Index (AHI) is an index used to indicate the severity of sleep apnea during a sleep session. The AHI is calculated by dividing the number of apnea and/or hypopnea events experienced by the user during the sleep session by the total number of hours of sleep in the sleep session. The event can be, for example, a pause in breathing that lasts for at least 10 seconds. An AHI that is less than 5 is considered normal. An AHI that is greater than or equal to 5, but less than 15 is considered indicative of mild sleep apnea. An AHI that is greater than or equal to 15, but less than 30 is considered indicative of moderate sleep apnea. An AHI that is greater than or equal to 30 is considered indicative of severe sleep apnea. In children, an AHI that is greater than 1 is considered abnormal. Sleep apnea can be considered “controlled” when the AHI is normal, or when the AHI is normal or mild. The AHI can also be used in combination with oxygen desaturation levels to indicate the severity of Obstructive Sleep Apnea.

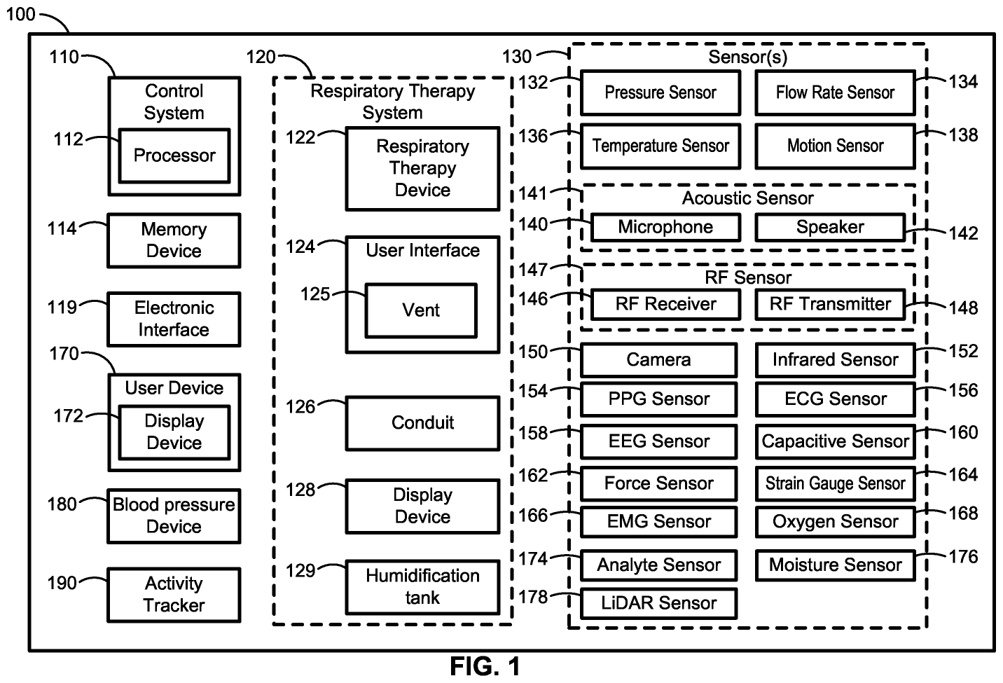

Referring to FIG. 1, a system 100, according to some implementations of the present disclosure, is illustrated. The system 100 includes a control system 110, a memory device 114, an electronic interface 119, one or more sensors 130, and one or more user devices 170. In some implementations, the system 100 further optionally includes a respiratory therapy system 120 (that includes a respiratory therapy device 122), a blood pressure device 180, an activity tracker 190, or any combination thereof. In further implementations, the system 100 optionally includes a respiratory therapy system 120 (that includes a respiratory therapy device 122), a blood pressure device 180, an activity tracker 190, or any combination thereof.

The control system 110 includes one or more processors 112 (hereinafter, processor 112). The control system 110 is generally used to control (e.g., actuate) the various components of the system 100 and/or analyze data obtained and/or generated by the components of the system 100. The processor 112 can be a general or special purpose processor or microprocessor. While one processor 112 is shown in FIG. 1, the control system 110 can include any suitable number of processors (e.g., one processor, two processors, five processors, ten processors, etc.) that can be in a single housing, or located remotely from each other. The control system 110 can be coupled to and/or positioned within, for example, a housing of the user device 170, and/or within a housing of one or more of the sensors 130. The control system 110 can be centralized (within one such housing) or decentralized (within two or more of such housings, which are physically distinct). In such implementations including two or more housings containing the control system 110, such housings can be located proximately and/or remotely from each other.

The memory device 114 stores machine-readable instructions that are executable by the processor 112 of the control system 110. The memory device 114 can be any suitable computer readable storage device or media, such as, for example, a random or serial access memory device, a hard drive, a solid state drive, a flash memory device, etc. While one memory device 114 is shown in FIG. 1, the system 100 can include any suitable number of memory devices 114 (e.g., one memory device, two memory devices, five memory devices, ten memory devices, etc.). The memory device 114 can be coupled to and/or positioned within a housing of the respiratory therapy device 122, within a housing of the user device 170, within a housing of one or more of the sensors 130, or any combination thereof. Like the control system 110, the memory device 114 can be centralized (within one such housing) or decentralized (within two or more of such housings, which are physically distinct).

In some implementations, the memory device 114 stores a user profile associated with a user. The user profile can include, for example, demographic information associated with the user, biometric information associated with the user, medical information associated with the user, self-reported user feedback, sleep parameters associated with the user (e.g., sleep-related parameters recorded from one or more earlier sleep sessions), or any combination thereof. The demographic information can include, for example, information indicative of an age of the user, a gender of the user, a race of the user, a geographic location of the user, a relationship status, a family history of insomnia or sleep apnea, an employment status of the user, an educational status of the user, a socioeconomic status of the user, or any combination thereof. The medical information can include, for example, information indicative of one or more medical conditions associated with the user, medication usage by the user, or both. The medical information data can further include a multiple sleep latency test (MSLT) result or score, and/or a Pittsburgh Sleep Quality Index (PSQI) score or value, and/or an Epworth Sleepiness Scale (ESS) assessment. The self-reported user feedback can include information indicative of a self-reported subjective sleep score (e.g., poor, average, excellent), a self-reported subjective stress level of the user, a self-reported subjective fatigue level of the user, a self-reported subjective health status of the user, a recent life event experienced by the user, or any combination thereof.

The electronic interface 119 is configured to receive data (e.g., physiological data and/or acoustic data) from the one or more sensors 130 such that the data can be stored in the memory device 114 and/or analyzed by the processor 112 of the control system 110. The electronic interface 119 can communicate with the one or more sensors 130 using a wired connection or a wireless connection (e.g., using an RF communication protocol, a WiFi communication protocol, a Bluetooth communication protocol, over a cellular network, etc.). The electronic interface 119 can include an antenna, a receiver (e.g., an RF receiver), a transmitter (e.g., an RF transmitter), a transceiver, or any combination thereof. The electronic interface 119 can also include one more processors and/or one more memory devices that are the same as, or similar to, the processor 112 and the memory device 114 described herein. In some implementations, the electronic interface 119 is coupled to or integrated in the user device 170. In other implementations, the electronic interface 119 is coupled to or integrated (e.g., in a housing) with the control system 110 and/or the memory device 114.

As noted above, in some implementations, the system 100 optionally includes a respiratory therapy system 120. The respiratory therapy system 120 can include a respiratory pressure therapy device (RPT) 122 (referred to herein as respiratory therapy device 122), a user interface 124, a conduit 126 (also referred to as a tube or an air circuit), a display device 128, a humidification tank 129, or any combination thereof. In some implementations, the control system 110, the memory device 114, the display device 128, one or more of the sensors 130, and the humidification tank 129 are part of the respiratory therapy device 122. Respiratory pressure therapy refers to the application of a supply of air to an entrance of the user's airways at a controlled target pressure that is nominally positive with respect to atmosphere throughout the user's breathing cycle (e.g., in contrast to negative pressure therapies such as the tank ventilator or cuirass). The respiratory therapy system 120 is generally used to treat individuals suffering from one or more sleep-related respiratory disorders (e.g., obstructive sleep apnea, central sleep apnea, or mixed sleep apnea).

The respiratory therapy device 122 has a blower motor (not shown) that is generally used to generate pressurized air that is delivered to the user (e.g., using one or more motors that drive one or more compressors). In some implementations, the respiratory therapy device 122 generates continuous constant air pressure that is delivered to the user. In other implementations, the respiratory therapy device 122 generates two or more predetermined pressures (e.g., a first predetermined air pressure and a second predetermined air pressure). In still other implementations, the respiratory therapy device 122 is configured to generate a variety of different air pressures within a predetermined range. For example, the respiratory therapy device 122 can deliver at least about 6 cm H₂O, at least about 10 cm H₂O, at least about 20 cm H₂O, between about 6 cm H₂O and about 10 cm H₂O, between about 7 cm H₂O and about 12 cm H₂O, etc. The respiratory therapy device 122 can also deliver pressurized air at a predetermined flow rate between, for example, about −20 L/min and about 150 L/min, while maintaining a positive pressure (relative to the ambient pressure).

The user interface 124 engages a portion of the user's face and delivers pressurized air from the respiratory therapy device 122 to the user's airway to aid in preventing the airway from narrowing and/or collapsing during sleep. This may also increase the user's oxygen intake during sleep. Generally, the user interface 124 engages the user's face such that the pressurized air is delivered to the user's airway via the user's mouth, the user's nose, or both the user's mouth and nose. Together, the respiratory therapy device 122, the user interface 124, and the conduit 126 form an air pathway fluidly coupled with an airway of the user. The pressurized air also increases the user's oxygen intake during sleep. Depending upon the therapy to be applied, the user interface 124 may form a seal, for example, with a region or portion of the user's face, to facilitate the delivery of air at a pressure at sufficient variance with ambient pressure to effect therapy, for example, at a positive pressure of about 10 cm H₂O relative to ambient pressure. For other forms of therapy, such as the delivery of oxygen, the user interface may not include a seal sufficient to facilitate delivery to the airways of a supply of gas at a positive pressure of about 10 cm H₂O.

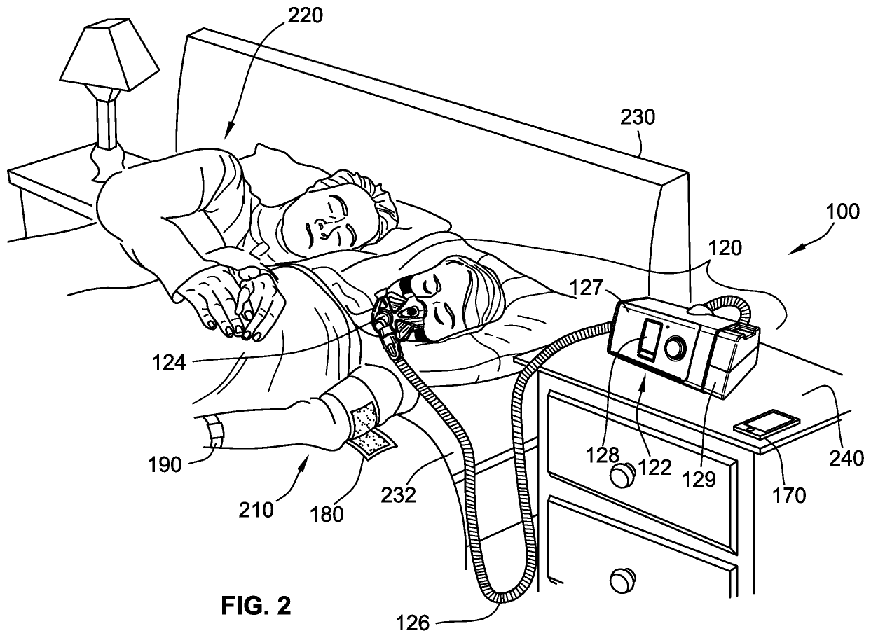

As shown in FIG. 2, in some implementations, the user interface 124 is a facial mask (e.g. a full facial mask) that covers the nose and mouth of the user 210. Alternatively, the user interface 124 can be a nasal mask that provides air to the nose of the user 210 or a nasal pillow mask that delivers air directly to the nostrils of the user 210. The user interface 124 can include a plurality of straps forming, for example, a headgear for aiding in positioning and/or stabilizing the interface on a portion of the user 210 (e.g., the face) and a conformal cushion (e.g., silicone, plastic, foam, etc.) that aids in providing an air-tight seal between the user interface 124 and the user 210. The user interface 124 can also include one or more vents 125 for permitting the escape of carbon dioxide and other gases exhaled by the user 210. In other implementations, the user interface 124 includes a mouthpiece (e.g., a night guard mouthpiece molded to conform to the teeth of the user 210, a mandibular repositioning device, etc.).

The conduit 126 (also referred to as an air circuit or tube) allows the flow of air between two components of a respiratory therapy system 120, such as the respiratory therapy device 122 and the user interface 124. In some implementations, there can be separate limbs of the conduit 126 for inhalation and exhalation. In other implementations, a single limb conduit is used for both inhalation and exhalation.

One or more of the respiratory therapy device 122, the user interface 124, the conduit 126, the display device 128, and the humidification tank 129 can contain one or more sensors (e.g., a pressure sensor, a flow rate sensor, or more generally any of the other sensors 130 described herein). These one or more sensors can be used, for example, to measure the air pressure and/or flow rate of pressurized air supplied by the respiratory therapy device 122.

The display device 128 is generally used to display image(s) including still images, video images, or both and/or information regarding the respiratory therapy device 122. For example, the display device 128 can provide information regarding the status of the respiratory therapy device 122 (e.g., whether the respiratory therapy device 122 is on/off, the pressure of the air being delivered by the respiratory therapy device 122, the temperature of the air being delivered by the respiratory therapy device 122, etc.) and/or other information (e.g., a sleep score and/or a therapy score, also referred to as a myAir™ score, such as described in WO 2016/061629, which is hereby incorporated by reference herein in its entirety; the current date/time; personal information for the user 210; etc.). In some implementations, the display device 128 acts as a human-machine interface (HMI) that includes a graphic user interface (GUI) configured to display the image(s) as an input interface. The display device 128 can be an LED display, an OLED display, an LCD display, or the like. The input interface can be, for example, a touchscreen or touch-sensitive substrate, a mouse, a keyboard, or any sensor system configured to sense inputs made by a human user interacting with the respiratory therapy device 122.

The humidification tank 129 is coupled to or integrated in the respiratory therapy device 122 and includes a reservoir of water that can be used to humidify the pressurized air delivered from the respiratory therapy device 122. The respiratory therapy device 122 can include one or more vents (not shown) and a heater to heat the water in the humidification tank 129 in order to humidify the pressurized air provided to the user 210. Additionally, in some implementations, the conduit 126 can also include a heating element (e.g., coupled to and/or imbedded in the conduit 126) that heats the pressurized air delivered to the user 210. The humidification tank 129 can be fluidly coupled to a water vapor inlet of the air pathway and deliver water vapor into the air pathway via the water vapor inlet, or can be formed in-line with the air pathway as part of the air pathway itself. In some implementations, the humidification tank 129 may not include the reservoir of water and thus waterless.

In some implementations, the system 100 can be used to deliver at least a portion of a substance from the receptacle (not shown) to the air pathway of the user based at least in part on the physiological data, the sleep-related parameters, other data or information, or any combination thereof. Generally, modifying the delivery of the portion of the substance into the air pathway can include (i) initiating the delivery of the substance into the air pathway, (ii) ending the delivery of the portion of the substance into the air pathway, (iii) modifying an amount of the substance delivered into the air pathway, (iv) modifying a temporal characteristic of the delivery of the portion of the substance into the air pathway, (v) modifying a quantitative characteristic of the delivery of the portion of the substance into the air pathway, (vi) modifying any parameter associated with the delivery of the substance into the air pathway, or (vii) a combination of (i)-(vi).

Modifying the temporal characteristic of the delivery of the portion of the substance into the air pathway can include changing the rate at which the substance is delivered, starting and/or finishing at different times, continuing for different time periods, changing the time distribution or characteristics of the delivery, changing the amount distribution independently of the time distribution, etc. The independent time and amount variation ensures that, apart from varying the frequency of the release of the substance, one can vary the amount of substance released each time. In this manner, a number of different combination of release frequencies and release amounts (e.g., higher frequency but lower release amount, higher frequency and higher amount, lower frequency and higher amount, lower frequency and lower amount, etc.) can be achieved. Other modifications to the delivery of the portion of the substance into the air pathway can also be utilized.

The respiratory therapy system 120 can be used, for example, as a ventilator or as a positive airway pressure (PAP) system, such as a continuous positive airway pressure (CPAP) system, an automatic positive airway pressure system (APAP), a bi-level or variable positive airway pressure system (BPAP or VPAP), or any combination thereof. The CPAP system delivers a predetermined amount of pressurized air (e.g., determined by a sleep physician) to the user 210. The APAP system automatically varies the pressurized air delivered to the user 210 based on, for example, respiration data associated with the user 210. The BPAP or VPAP system is configured to deliver a first predetermined pressure (e.g., an inspiratory positive airway pressure or IPAP) and a second predetermined pressure (e.g., an expiratory positive airway pressure or EPAP) that is lower than the first predetermined pressure.

Referring again to FIG. 2, a portion of the system 100 (FIG. 1), according to some implementations, is illustrated. The user 210 of the respiratory therapy system 120 and a bed partner 220 are located on a bed 230 and laying on a mattress 232. The user interface 124 (also referred to herein as a mask, e.g., a full facial mask) can be worn by the user 210 during a sleep session. The user interface 124 is fluidly coupled and/or connected to the respiratory therapy device 122 via the conduit 126. In turn, the respiratory therapy device 122 delivers pressurized air to the user 210 via the conduit 126 and the user interface 124 to increase the air pressure in the throat of the user 210 to aid in preventing the airway from closing and/or narrowing during sleep. The respiratory therapy device 122 can be positioned on a nightstand 240 that is directly adjacent to the bed 230 as shown in FIG. 2, or more generally, on any surface or structure that is generally adjacent to the bed 230 and/or the user 210.

Referring to back to FIG. 1, the one or more sensors 130 of the system 100 include a pressure sensor 132, a flow rate sensor 134, temperature sensor 136, a motion sensor 138, a microphone 140, a speaker 142, a radio-frequency (RF) receiver 146, a RF transmitter 148, a camera 150, an infrared sensor 152, a photoplethysmogram (PPG) sensor 154, an electrocardiogram (ECG) sensor 156, an electroencephalography (EEG) sensor 158, a capacitive sensor 160, a force sensor 162, a strain gauge sensor 164, an electromyography (EMG) sensor 166, an oxygen sensor 168, an analyte sensor 174, a moisture sensor 176, a LiDAR sensor 178, or any combination thereof. Generally, each of the one or more sensors 130 are configured to output sensor data that is received and stored in the memory device 114 or one or more other memory devices.

While the one or more sensors 130 are shown and described as including each of the pressure sensor 132, the flow rate sensor 134, the temperature sensor 136, the motion sensor 138, the microphone 140, the speaker 142, the RF receiver 146, the RF transmitter 148, the camera 150, the infrared sensor 152, the photoplethysmogram (PPG) sensor 154, the electrocardiogram (ECG) sensor 156, the electroencephalography (EEG) sensor 158, the capacitive sensor 160, the force sensor 162, the strain gauge sensor 164, the electromyography (EMG) sensor 166, the oxygen sensor 168, the analyte sensor 174, the moisture sensor 176, and the LiDAR sensor 178, more generally, the one or more sensors 130 can include any combination and any number of each of the sensors described and/or shown herein.

As described herein, the system 100 generally can be used to generate physiological data associated with a user (e.g., a user of the respiratory therapy system 120 shown in FIG. 2) during a sleep session. The physiological data can be analyzed to generate one or more sleep-related parameters, which can include any parameter, measurement, etc. related to the user during the sleep session. The one or more sleep-related parameters that can be determined for the user 210 during the sleep session include, for example, an Apnea-Hypopnea Index (AHI) score, a sleep score, a flow signal, a respiration signal, a respiration rate, an inspiration amplitude, an expiration amplitude, an inspiration-expiration ratio, a number of events per hour, a pattern of events, a stage, pressure settings of the respiratory therapy device 122, a heart rate, a heart rate variability, movement of the user 210, temperature, EEG activity, EMG activity, arousal, snoring, choking, coughing, whistling, wheezing, or any combination thereof.

The one or more sensors 130 can be used to generate, for example, physiological data, acoustic data, or both. Physiological data generated by one or more of the sensors 130 can be used by the control system 110 to determine a sleep-wake signal associated with the user 210 (FIG. 2) during the sleep session and one or more sleep-related parameters. The sleep-wake signal can be indicative of one or more sleep states, including wakefulness, relaxed wakefulness, micro-awakenings, or distinct sleep stages such as, for example, a rapid eye movement (REM) stage, a first non-REM stage (often referred to as “N1”), a second non-REM stage (often referred to as “N2”), a third non-REM stage (often referred to as “N3”), or any combination thereof. Methods for determining sleep states and/or sleep stages from physiological data generated by one or more sensors, such as the one or more sensors 130, are described in, for example, WO 2014/047310, US 2014/0088373, WO 2017/132726, WO 2019/122413, and WO 2019/122414, each of which is hereby incorporated by reference herein in its entirety.

In some implementations, the sleep-wake signal described herein can be timestamped to indicate a time that the user enters the bed, a time that the user exits the bed, a time that the user attempts to fall asleep, etc. The sleep-wake signal can be measured by the one or more sensors 130 during the sleep session at a predetermined sampling rate, such as, for example, one sample per second, one sample per 30 seconds, one sample per minute, etc. In some implementations, the sleep-wake signal can also be indicative of a respiration signal, a respiration rate, an inspiration amplitude, an expiration amplitude, an inspiration-expiration ratio, a number of events per hour, a pattern of events, pressure settings of the respiratory therapy device 122, or any combination thereof during the sleep session. The event(s) can include snoring, apneas, central apneas, obstructive apneas, mixed apneas, hypopneas, a mask leak (e.g., from the user interface 124), a restless leg, a sleeping disorder, choking, an increased heart rate, labored breathing, an asthma attack, an epileptic episode, a seizure, or any combination thereof. The one or more sleep-related parameters that can be determined for the user during the sleep session based on the sleep-wake signal include, for example, a total time in bed, a total sleep time, a sleep onset latency, a wake-after-sleep-onset parameter, a sleep efficiency, a fragmentation index, or any combination thereof. As described in further detail herein, the physiological data and/or the sleep-related parameters can be analyzed to determine one or more sleep-related scores. The physiological data, sleep-related parameters and/or sleep-related scores may be considered sleep quality metrics of an individual.

Physiological data and/or audio data generated by the one or more sensors 130 can also be used to determine a respiration signal associated with a user during a sleep session. The respiration signal is generally indicative of respiration or breathing of the user during the sleep session. The respiration signal can be indicative of and/or analyzed to determine (e.g., using the control system 110) one or more sleep-related parameters, such as, for example, a respiration rate, a respiration rate variability, an inspiration amplitude, an expiration amplitude, an inspiration-expiration ratio, an occurrence of one or more events, a number of events per hour, a pattern of events, a sleep state, a sleet stage, an apnea-hypopnea index (AHI), pressure settings of the respiratory therapy device 122, or any combination thereof. The one or more events can include snoring, apneas, central apneas, obstructive apneas, mixed apneas, hypopneas, a mask leak (e.g., from the user interface 124), a cough, a restless leg, a sleeping disorder, choking, an increased heart rate, labored breathing, an asthma attack, an epileptic episode, a seizure, increased blood pressure, or any combination thereof. Many of the described sleep-related parameters are physiological parameters, although some of the sleep-related parameters can be considered to be non-physiological parameters. Other types of physiological and/or non-physiological parameters can also be determined, either from the data from the one or more sensors 130, or from other types of data.

The pressure sensor 132 outputs pressure data that can be stored in the memory device 114 and/or analyzed by the processor 112 of the control system 110. In some implementations, the pressure sensor 132 is an air pressure sensor (e.g., barometric pressure sensor) that generates sensor data indicative of the respiration (e.g., inhaling and/or exhaling) of the user of the respiratory therapy system 120 and/or ambient pressure. In such implementations, the pressure sensor 132 can be coupled to or integrated in the respiratory therapy device 122. The pressure sensor 132 can be, for example, a capacitive sensor, an electromagnetic sensor, a piezoelectric sensor, a strain-gauge sensor, an optical sensor, a potentiometric sensor, or any combination thereof.

The flow rate sensor 134 outputs flow rate data that can be stored in the memory device 114 and/or analyzed by the processor 112 of the control system 110. Examples of flow rate sensors (such as, for example, the flow rate sensor 134) are described in International Publication No. WO 2012/012835, which is hereby incorporated by reference herein in its entirety. In some implementations, the flow rate sensor 134 is used to determine an air flow rate from the respiratory therapy device 122, an air flow rate through the conduit 126, an air flow rate through the user interface 124, or any combination thereof. In such implementations, the flow rate sensor 134 can be coupled to or integrated in the respiratory therapy device 122, the user interface 124, or the conduit 126. The flow rate sensor 134 can be a mass flow rate sensor such as, for example, a rotary flow meter (e.g., Hall effect flow meters), a turbine flow meter, an orifice flow meter, an ultrasonic flow meter, a hot wire sensor, a vortex sensor, a membrane sensor, or any combination thereof. In some implementations, the flow rate sensor 134 is configured to measure a vent flow (e.g., intentional “leak”), an unintentional leak (e.g., mouth leak and/or mask leak), a patient flow (e.g., air into and/or out of lungs), or any combination thereof. In some implementations, the flow rate data can be analyzed to determine cardiogenic oscillations of the user. In one example, the pressure sensor 132 can be used to determine a blood pressure of a user.

The temperature sensor 136 outputs temperature data that can be stored in the memory device 114 and/or analyzed by the processor 112 of the control system 110. In some implementations, the temperature sensor 136 generates temperatures data indicative of a core body temperature of the user 210 (FIG. 2), a skin temperature of the user 210, a temperature of the air flowing from the respiratory therapy device 122 and/or through the conduit 126, a temperature in the user interface 124, an ambient temperature, or any combination thereof. The temperature sensor 136 can be, for example, a thermocouple sensor, a thermistor sensor, a silicon band gap temperature sensor or semiconductor-based sensor, a resistance temperature detector, or any combination thereof.

The motion sensor 138 outputs motion data that can be stored in the memory device 114 and/or analyzed by the processor 112 of the control system 110. The motion sensor 138 can be used to detect movement of the user 210 during the sleep session, and/or detect movement of any of the components of the respiratory therapy system 120, such as the respiratory therapy device 122, the user interface 124, or the conduit 126. The motion sensor 138 can include one or more inertial sensors, such as accelerometers, gyroscopes, and magnetometers. In some implementations, the motion sensor 138 alternatively or additionally generates one or more signals representing bodily movement of the user, from which may be obtained a signal representing a sleep state of the user; for example, via a respiratory movement of the user. In some implementations, the motion data from the motion sensor 138 can be used in conjunction with additional data from another sensor 130 to determine the sleep state of the user.

The microphone 140 outputs sound and/or audio data that can be stored in the memory device 114 and/or analyzed by the processor 112 of the control system 110. The audio data generated by the microphone 140 is reproducible as one or more sound(s) during a sleep session (e.g., sounds from the user 210). The audio data form the microphone 140 can also be used to identify (e.g., using the control system 110) an event experienced by the user during the sleep session, as described in further detail herein. The microphone 140 can be coupled to or integrated in the respiratory therapy device 122, the user interface 124, the conduit 126, or the user device 170. In some implementations, the system 100 includes a plurality of microphones (e.g., two or more microphones and/or an array of microphones with beamforming) such that sound data generated by each of the plurality of microphones can be used to discriminate the sound data generated by another of the plurality of microphones.

The speaker 142 outputs sound waves that are audible to a user of the system 100 (e.g., the user 210 of FIG. 2). The speaker 142 can be used, for example, as an alarm clock or to play an alert or message to the user 210 (e.g., in response to an event). In some implementations, the speaker 142 can be used to communicate the audio data generated by the microphone 140 to the user. The speaker 142 can be coupled to or integrated in the respiratory therapy device 122, the user interface 124, the conduit 126, or the user device 170.

The microphone 140 and the speaker 142 can be used as separate devices. In some implementations, the microphone 140 and the speaker 142 can be combined into an acoustic sensor 141 (e.g., a SONAR sensor), as described in, for example, WO 2018/050913 and WO 2020/104465, each of which is hereby incorporated by reference herein in its entirety. In such implementations, the speaker 142 generates or emits sound waves at a predetermined interval and the microphone 140 detects the reflections of the emitted sound waves from the speaker 142. The sound waves generated or emitted by the speaker 142 have a frequency that is not audible to the human ear (e.g., below 20 Hz or above around 18 kHz) so as not to disturb the sleep of the user 210 or the bed partner 220 (FIG. 2). Based at least in part on the data from the microphone 140 and/or the speaker 142, the control system 110 can determine a location of the user 210 (FIG. 2) and/or one or more of the sleep-related parameters described in herein such as, for example, a respiration signal, a respiration rate, an inspiration amplitude, an expiration amplitude, an inspiration-expiration ratio, a number of events per hour, a pattern of events, a sleep state, a sleep stage, pressure settings of the respiratory therapy device 122, or any combination thereof. In such a context, a sonar sensor may be understood to concern an active acoustic sensing, such as by generating and/or transmitting ultrasound and/or low frequency ultrasound sensing signals (e.g., in a frequency range of about 17-23 kHz, 18-22 kHz, or 17-18 kHz, for example), through the air. Such a system may be considered in relation to WO 2018/050913 and WO 2020/104465 mentioned above, each of which is hereby incorporated by reference herein in its entirety.

In some implementations, the sensors 130 include (i) a first microphone that is the same as, or similar to, the microphone 140, and is integrated in the acoustic sensor 141 and (ii) a second microphone that is the same as, or similar to, the microphone 140, but is separate and distinct from the first microphone that is integrated in the acoustic sensor 141.

The RF transmitter 148 generates and/or emits radio waves having a predetermined frequency and/or a predetermined amplitude (e.g., within a high frequency band, within a low frequency band, long wave signals, short wave signals, etc.). The RF receiver 146 detects the reflections of the radio waves emitted from the RF transmitter 148, and this data can be analyzed by the control system 110 to determine a location of the user 210 (FIG. 2) and/or one or more of the sleep-related parameters described herein. An RF receiver (either the RF receiver 146 and the RF transmitter 148 or another RF pair) can also be used for wireless communication between the control system 110, the respiratory therapy device 122, the one or more sensors 130, the user device 170, or any combination thereof. While the RF receiver 146 and RF transmitter 148 are shown as being separate and distinct elements in FIG. 1, in some implementations, the RF receiver 146 and RF transmitter 148 are combined as a part of an RF sensor 147 (e.g. a RADAR sensor). In some such implementations, the RF sensor 147 includes a control circuit. The specific format of the RF communication can be Wi-Fi, Bluetooth, or the like.

In some implementations, the RF sensor 147 is a part of a mesh system. One example of a mesh system is a Wi-Fi mesh system, which can include mesh nodes, mesh router(s), and mesh gateway(s), each of which can be mobile/movable or fixed. In such implementations, the Wi-Fi mesh system includes a Wi-Fi router and/or a Wi-Fi controller and one or more satellites (e.g., access points), each of which include an RF sensor that the is the same as, or similar to, the RF sensor 147. The Wi-Fi router and satellites continuously communicate with one another using Wi-Fi signals. The Wi-Fi mesh system can be used to generate motion data based on changes in the Wi-Fi signals (e.g., differences in received signal strength) between the router and the satellite(s) due to an object or person moving partially obstructing the signals. The motion data can be indicative of motion, breathing, heart rate, gait, falls, behavior, etc., or any combination thereof.

The camera 150 outputs image data reproducible as one or more images (e.g., still images, video images, thermal images, or any combination thereof) that can be stored in the memory device 114. The image data from the camera 150 can be used by the control system 110 to determine one or more of the sleep-related parameters described herein, such as, for example, one or more events (e.g., periodic limb movement or restless leg syndrome), a respiration signal, a respiration rate, an inspiration amplitude, an expiration amplitude, an inspiration-expiration ratio, a number of events per hour, a pattern of events, a sleep state, a sleep stage, or any combination thereof. Further, the image data from the camera 150 can be used to, for example, identify a location of the user, to determine chest movement of the user 210 (FIG. 2), to determine air flow of the mouth and/or nose of the user 210, to determine a time when the user 210 enters the bed 230 (FIG. 2), and to determine a time when the user 210 exits the bed 230. In some implementations, the camera 150 includes a wide angle lens or a fish eye lens.

The infrared (IR) sensor 152 outputs infrared image data reproducible as one or more infrared images (e.g., still images, video images, or both) that can be stored in the memory device 114. The infrared data from the IR sensor 152 can be used to determine one or more sleep-related parameters during a sleep session, including a temperature of the user 210 and/or movement of the user 210. The IR sensor 152 can also be used in conjunction with the camera 150 when measuring the presence, location, and/or movement of the user 210. The IR sensor 152 can detect infrared light having a wavelength between about 700 nm and about 1 mm, for example, while the camera 150 can detect visible light having a wavelength between about 380 nm and about 740 nm.

The PPG sensor 154 outputs physiological data associated with the user 210 (FIG. 2) that can be used to determine one or more sleep-related parameters, such as, for example, a heart rate, a heart rate variability, a cardiac cycle, respiration rate, an inspiration amplitude, an expiration amplitude, an inspiration-expiration ratio, estimated blood pressure parameter(s), or any combination thereof. The PPG sensor 154 can be worn by the user 210, embedded in clothing and/or fabric that is worn by the user 210, embedded in and/or coupled to the user interface 124 and/or its associated headgear (e.g., straps, etc.), etc. A PAT (peripheral arterial tone) sensing device may make use of a fingertip mounted PPG probe, e.g. PPG sensor 154. The PPG probe operates with an optical technology that detects blood volume changes in the tissue's microvascular bed. As noted above, PPG measurements are used to derive the arterial blood oxygen saturation (SpO₂), pulse rate (PR), and changes in peripheral arterial tone, which are then used to detect respiratory events. Peripheral arterial tone refers to the tone of the peripheral arterial smooth muscle tissue. When the muscle tone of peripheral arteries increases, the arteries' diameter decreases, resulting in a reduction of perfusion and thus a decrease in pulsatile blood volume in the peripheral tissue. The decrease in pulsatile blood volume in the peripheral tissue is picked up as a drop in the PPG signal swing between systole and diastole.

The PAT signal may be derived from the PPG signal from the PPG sensor, such as by the method described in PCT/EP2021/067532 (Pub. No. WO2021/260190A1), the disclosure of which is incorporated by reference herein in its entirety. The PPG-derived signal, which may be derived by trending such pulsatile blood volume reductions, is referred to as the PAT signal.

The ECG sensor 156 outputs physiological data associated with electrical activity of the heart of the user 210. In some implementations, the ECG sensor 156 includes one or more electrodes that are positioned on or around a portion of the user 210 during the sleep session. The physiological data from the ECG sensor 156 can be used, for example, to determine one or more of the sleep-related parameters described herein.

The EEG sensor 158 outputs physiological data associated with electrical activity of the brain of the user 210. In some implementations, the EEG sensor 158 includes one or more electrodes that are positioned on or around the scalp of the user 210 during the sleep session. The physiological data from the EEG sensor 158 can be used, for example, to determine a sleep state and/or a sleep stage of the user 210 at any given time during the sleep session. In some implementations, the EEG sensor 158 can be integrated in the user interface 124 and/or the associated headgear (e.g., straps, etc.).

The capacitive sensor 160, the force sensor 162, and the strain gauge sensor 164 output data that can be stored in the memory device 114 and used by the control system 110 to determine one or more of the sleep-related parameters described herein. The EMG sensor 166 outputs physiological data associated with electrical activity produced by one or more muscles. The oxygen sensor 168 outputs oxygen data indicative of an oxygen concentration of gas (e.g., in the conduit 126 or at the user interface 124). The oxygen sensor 168 can be, for example, an ultrasonic oxygen sensor, an electrical oxygen sensor, a chemical oxygen sensor, an optical oxygen sensor, a pulse oximeter (e.g., SpO₂ sensor), or any combination thereof. In some implementations, the one or more sensors 130 also include a galvanic skin response (GSR) sensor, a blood flow sensor, a respiration sensor, a pulse sensor, a sphygmomanometer sensor, an oximetry sensor, or any combination thereof.

The analyte sensor 174 can be used to detect the presence of an analyte in the exhaled breath of the user 210. The data output by the analyte sensor 174 can be stored in the memory device 114 and used by the control system 110 to determine the identity and concentration of any analytes in the breath of the user 210. In some implementations, the analyte sensor 174 is positioned near a mouth of the user 210 to detect analytes in breath exhaled from the user 210's mouth. For example, when the user interface 124 is a facial mask that covers the nose and mouth of the user 210, the analyte sensor 174 can be positioned within the facial mask to monitor the user 210's mouth breathing. In other implementations, such as when the user interface 124 is a nasal mask or a nasal pillow mask, the analyte sensor 174 can be positioned near the nose of the user 210 to detect analytes in breath exhaled through the user's nose. In still other implementations, the analyte sensor 174 can be positioned near the user 210's mouth when the user interface 124 is a nasal mask or a nasal pillow mask. In this implementation, the analyte sensor 174 can be used to detect whether any air is inadvertently leaking from the user 210's mouth. In some implementations, the analyte sensor 174 is a volatile organic compound (VOC) sensor that can be used to detect carbon-based chemicals or compounds. In some implementations, the analyte sensor 174 can also be used to detect whether the user 210 is breathing through their nose or mouth. For example, if the data output by an analyte sensor 174 positioned near the mouth of the user 210 or within the facial mask (in implementations where the user interface 124 is a facial mask) detects the presence of an analyte, the control system 110 can use this data as an indication that the user 210 is breathing through their mouth.

The moisture sensor 176 outputs data that can be stored in the memory device 114 and used by the control system 110. The moisture sensor 176 can be used to detect moisture in various areas surrounding the user (e.g., inside the conduit 126 or the user interface 124, near the user 210's face, near the connection between the conduit 126 and the user interface 124, near the connection between the conduit 126 and the respiratory therapy device 122, etc.). Thus, in some implementations, the moisture sensor 176 can be coupled to or integrated in the user interface 124 or in the conduit 126 to monitor the humidity of the pressurized air from the respiratory therapy device 122. In other implementations, the moisture sensor 176 is placed near any area where moisture levels need to be monitored. The moisture sensor 176 can also be used to monitor the humidity of the ambient environment surrounding the user 210, for example, the air inside the bedroom.

The Light Detection and Ranging (LiDAR) sensor 178 can be used for depth sensing. This type of optical sensor (e.g., laser sensor) can be used to detect objects and build three dimensional (3D) maps of the surroundings, such as of a living space. LiDAR can generally utilize a pulsed laser to make time of flight measurements. LiDAR is also referred to as 3D laser scanning. In an example of use of such a sensor, a fixed or mobile device (such as a smartphone) having a LiDAR sensor 166 can measure and map an area extending 5 meters or more away from the sensor. The LiDAR data can be fused with point cloud data estimated by an electromagnetic RADAR sensor, for example. The LiDAR sensor(s) 178 can also use artificial intelligence (AI) to automatically geofence RADAR systems by detecting and classifying features in a space that might cause issues for RADAR systems, such a glass windows (which can be highly reflective to RADAR). LiDAR can also be used to provide an estimate of the height of a person, as well as changes in height when the person sits down, or falls down, for example. LiDAR may be used to form a 3D mesh representation of an environment. In a further use, for solid surfaces through which radio waves pass (e.g., radio-translucent materials), the LiDAR may reflect off such surfaces, thus allowing a classification of different type of obstacles.

In some implementations, the one or more sensors 130 also include a galvanic skin response (GSR) sensor, a blood flow sensor, a respiration sensor, a pulse sensor, a sphygmomanometer sensor, an oximetry sensor, a sonar sensor, a RADAR sensor, a blood glucose sensor, a color sensor, a pH sensor, an air quality sensor, a tilt sensor, a rain sensor, a soil moisture sensor, a water flow sensor, an alcohol sensor, or any combination thereof.

While shown separately in FIG. 1, any combination of the one or more sensors 130 can be integrated in and/or coupled to any one or more of the components of the system 100, including the respiratory therapy device 122, the user interface 124, the conduit 126, the humidification tank 129, the control system 110, the user device 170, the activity tracker 180, or any combination thereof. For example, the microphone 140 and the speaker 142 can be integrated in and/or coupled to the user device 170 and the pressure sensor 130 and/or flow rate sensor 132 are integrated in and/or coupled to the respiratory therapy device 122. In some implementations, at least one of the one or more sensors 130 is not coupled to the respiratory therapy device 122, the control system 110, or the user device 170, and is positioned generally adjacent to the user 210 during the sleep session (e.g., positioned on or in contact with a portion of the user 210, worn by the user 210, coupled to or positioned on the nightstand, coupled to the mattress, coupled to the ceiling, etc.).

The data from the one or more sensors 130 can be analyzed to determine one or more sleep-related parameters, which can include a respiration signal, a respiration rate, a respiration pattern, an inspiration amplitude, an expiration amplitude, an inspiration-expiration ratio, an occurrence of one or more events, a number of events per hour, a pattern of events, a sleep state, an apnea-hypopnea index (AHI), or any combination thereof. The one or more events can include snoring, apneas, central apneas, obstructive apneas, mixed apneas, hypopneas, a mask leak, a cough, a restless leg, a sleeping disorder, choking, an increased heart rate, labored breathing, an asthma attack, an epileptic episode, a seizure, increased blood pressure, or any combination thereof. Many of these sleep-related parameters are physiological parameters, although some of the sleep-related parameters can be considered to be non-physiological parameters. Other types of physiological and non-physiological parameters can also be determined, either from the data from the one or more sensors 130, or from other types of data.

The user device 170 (FIG. 1) includes a display device 172. The user device 170 can be, for example, a mobile device such as a smart phone, a tablet, a gaming console, a smart watch, a laptop, or the like. Alternatively, the user device 170 can be an external sensing system, a television (e.g., a smart television) or another smart home device (e.g., a smart speaker(s) such as Google Home™, Google Nest™, Amazon Echo™, Amazon Echo Show™ Alexa™-enabled devices, etc.). In some implementations, the user device is a wearable device (e.g., a smart watch). The display device 172 is generally used to display image(s) including still images, video images, or both. In some implementations, the display device 172 acts as a human-machine interface (HMI) that includes a graphic user interface (GUI) configured to display the image(s) and an input interface. The display device 172 can be an LED display, an OLED display, an LCD display, or the like. The input interface can be, for example, a touchscreen or touch-sensitive substrate, a mouse, a keyboard, or any sensor system configured to sense inputs made by a human user interacting with the user device 170. In some implementations, one or more user devices can be used by and/or included in the system 100.

The blood pressure device 180 is generally used to aid in generating cardiovascular data for determining one or more blood pressure measurements associated with the user 210. The blood pressure device 180 can include at least one of the one or more sensors 130 to measure, for example, a systolic blood pressure component and/or a diastolic blood pressure component.

In some implementations, the blood pressure device 180 is a sphygmomanometer including an inflatable cuff that can be worn by the user 210 and a pressure sensor (e.g., the pressure sensor 132 described herein). For example, as shown in the example of FIG. 2, the blood pressure device 180 can be worn on an upper arm of the user 210. In such implementations where the blood pressure device 180 is a sphygmomanometer, the blood pressure device 180 also includes a pump (e.g., a manually operated bulb) for inflating the cuff. In some implementations, the blood pressure device 180 is physically and/or communicatively coupled to the respiratory therapy device 122 of the respiratory therapy system 120, which in turn delivers or triggers delivery pressurized air to inflate the cuff. More generally, the blood pressure device 180 can be communicatively coupled to, and/or optionally physically integrated with (e.g., within a housing) the respiratory therapy system 120. Additionally, or alternatively, the blood pressure device 180 can be communicatively coupled to the control system 110, the memory device 114, the user device 170, and/or the activity tracker 190, which are in turn communicatively coupled to the respiratory therapy system 120.

In some implementations, the blood pressure device 180 is an invasive device which can continuously monitor arterial blood pressure of the user 210 and take an arterial blood sample on demand for analyzing a gas content of the arterial blood. In other implementations, the blood pressure device 180 is a non-invasive continuous blood pressure monitor that uses a radio frequency (RF) sensor such as a Radio Detection and Ranging (RADAR) sensor, a Sound Navigation and Ranging (SONAR) sensor, an infrared (IR) sensor, a pressure sensor, a displacement sensor, or a combination thereof. The RF sensor is capable of measuring blood pressure of the user 210 once very few seconds (e.g. 3 seconds, 5 seconds, 7 seconds, etc.) The RF sensor may use a continuous wave; a frequency-modulated continuous wave (FMCW) with ramp chirp, triangle, sinewave, and other modulation schemes such as phase-shift keying (PSK), frequency shift keying (FSK) etc.; a pulsed continuous wave; and/or a wave spread in ultra wideband (UWB) ranges (which may include spreading, Pseudo Random Noise (PRN) codes or impulse systems). In implementations, suitable RF/RADAR-based sensors include those developed by Blumio™ and Infineon™, which sensor is a wearable, non-invasive blood pressure sensor based on Infineon™'s XENSIV radar chipset.

When using the RADAR sensor or the SONAR sensor, a mattress on the bed 230 can calculate Ballistocardiography (BCG), and an optical sensor located on the body of the user 210 (e.g., smartwatch, smartpatch, etc.) or remotely (e.g. video camera) can calculate Photoplethysmography (PPG), in some implementations. The BCG and PPG values can then be used to measure a time delay between these two signals in order to calculate both systolic blood pressure and diastolic blood pressure.

In some implementations, the PPG with auto gain and signal to noise ratio (SNR) management can be used to calculate pulse transit time (PTT), pulse wave analysis, and with appropriate calibration parameters (either demographic or personalized) can be used to estimate the blood pressure of the user 210. For example, an optical sensor can emit coherent light into the skin of the user 210, and then collect and capture the reflected light from the red blood cells in the blood vessels in the skin under the optical sensor. Thus, the optical sensor and associated software is capable of detecting the pulse wave to determine a measurement of the blood pressure of the user 210. Other techniques can use video directly, such as using transdermal optical imaging (e.g., via a customized camera system or via a smartphone) to measure blood pressure from a video of the user's face (such as with ambient light, or a light such as a LED or infrared source). Yet other sensors can include ultrasonic sensors, whereby pulses and return echoes are used to map the anterior and posterior walls of the artery.

In still other implementations, the blood pressure device 180 is an ambulatory blood pressure monitor communicatively coupled to the respiratory therapy system 120. An ambulatory blood pressure monitor may include a portable recording device attached to a belt or strap worn by the user 210 and an inflatable cuff attached to the portable recording device and worn around an arm of the user 210. The ambulatory blood pressure monitor is configured to measure blood pressure periodically, such as between about every fifteen minutes to about thirty minutes over a 24-hour or a 48-hour period. The ambulatory blood pressure monitor may measure heart rate of the user 210 at the same time. These multiple readings may be averaged over the 24-hour period. The ambulatory blood pressure monitor determines any changes in the measured blood pressure and heart rate of the user 210, as well as any distribution and/or trending patterns of the blood pressure and heart rate data during a sleeping period and an awakened period of the user 210. The measured data and statistics may then be communicated to the respiratory therapy system 120.

The activity tracker 190 is generally used to aid in generating physiological data for determining an activity measurement associated with the user 210. The activity tracker 190 can include one or more of the sensors 130 described herein, such as, for example, the motion sensor 138 (e.g., one or more accelerometers and/or gyroscopes), the PPG sensor 154, and/or the ECG sensor 156. The physiological data from the activity tracker 190 can be used to determine, for example, a number of steps, a distance traveled, a number of steps climbed, a duration of physical activity, a type of physical activity, an intensity of physical activity, time spent standing, a respiration rate, an average respiration rate, a resting respiration rate, a maximum respiration rate, a respiration rate variability, a heart rate, an average heart rate, a resting heart rate, a maximum heart rate, a heart rate variability, a number of calories burned, blood oxygen saturation, electrodermal activity (also known as skin conductance or galvanic skin response), or any combination thereof. In some implementations, the activity tracker 190 is coupled (e.g., electronically or physically) to the user device 170.

In some implementations, the activity tracker 190 is a wearable device that can be worn by the user 210, such as a smartwatch, a wristband, a ring, or a patch. For example, referring to FIG. 2, the activity tracker 190 is worn on a wrist of the user 210. The activity tracker 190 can also be coupled to or integrated a garment or clothing that is worn by the user 210. Alternatively, still, the activity tracker 190 can also be coupled to or integrated in (e.g., within the same housing) the user device 170. More generally, the activity tracker 190 can be communicatively coupled with, or physically integrated in (e.g., within a housing), the control system 110, the memory device 114, the respiratory therapy system 120, the user device 170, and/or the blood pressure device 180. In some implementations, the activity tracker 190 can comprise the blood pressure monitor 180, such as exemplified by Samsung™'s Galaxy Watch3 or Galaxy Watch Active2 smartwatches which can generate the blood pressure measurement by using pulse wave analysis.

While the control system 110 and the memory device 114 are described and shown in FIG. 1 as being a separate and distinct component of the system 100, in some implementations, the control system 110 and/or the memory device 114 are integrated in the user device 170 and/or the respiratory therapy device 122. Alternatively, in some implementations, the control system 110 or a portion thereof (e.g., the processor 112) can be located in a cloud (e.g., integrated in a server, integrated in an Internet of Things (IoT) device, connected to the cloud, be subject to edge cloud processing, etc.), located in one or more servers (e.g., remote servers, local servers, etc., or any combination thereof.

While system 100 is shown as including all of the components described above, more or fewer components can be included in a system according to implementations of the present disclosure. For example, a first alternative system includes the control system 110, the memory device 114, and at least one of the one or more sensors 130 and does not include the respiratory therapy system 120. As another example, a second alternative system includes the control system 110, the memory device 114, at least one of the one or more sensors 130, and the user device 170. As yet another example, a third alternative system includes the control system 110, the memory device 114, the respiratory therapy system 120, at least one of the one or more sensors 130, and the user device 170. Thus, various systems can be formed using any portion or portions of the components shown and described herein and/or in combination with one or more other components.

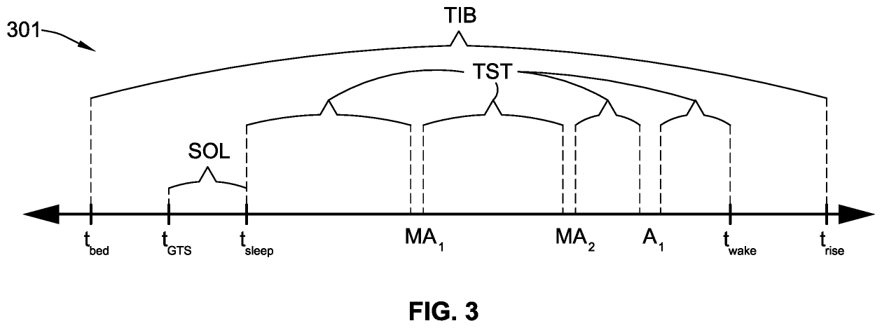

As used herein, a sleep session can be defined in a number of ways based on, for example, an initial start time and an end time. Referring to FIG. 3, an exemplary timeline 300 for a sleep session is illustrated. The timeline 300 includes an enter bed time (t_{bed}), a go-to-sleep time (t_{GTS}), an initial sleep time (tₛₗₑₑₚ), a first micro-awakening MA₁ and a second micro-awakening MA₂, a wake-up time (t_{wake}), and a rising time (tᵣᵢₛₑ).

As used herein, a sleep session can be defined in multiple ways. For example, a sleep session can be defined by an initial start time and an end time. In some implementations, a sleep session is a duration where the user is asleep, that is, the sleep session has a start time and an end time, and during the sleep session, the user does not wake until the end time. That is, any period of the user being awake is not included in a sleep session. From this first definition of sleep session, if the user wakes ups and falls asleep multiple times in the same night, each of the sleep intervals separated by an awake interval is a sleep session.

Alternatively, in some implementations, a sleep session has a start time and an end time, and during the sleep session, the user can wake up, without the sleep session ending, so long as a continuous duration that the user is awake is below an awake duration threshold. The awake duration threshold can be defined as a percentage of a sleep session. The awake duration threshold can be, for example, about twenty percent of the sleep session, about fifteen percent of the sleep session duration, about ten percent of the sleep session duration, about five percent of the sleep session duration, about two percent of the sleep session duration, etc., or any other threshold percentage. In some implementations, the awake duration threshold is defined as a fixed amount of time, such as, for example, about one hour, about thirty minutes, about fifteen minutes, about ten minutes, about five minutes, about two minutes, etc., or any other amount of time.

In some implementations, a sleep session is defined as the entire time between the time in the evening at which the user first entered the bed, and the time the next morning when user last left the bed. Put another way, a sleep session can be defined as a period of time that begins on a first date (e.g., Monday, Jan. 6, 2020) at a first time (e.g., 10:00 PM), that can be referred to as the current evening, when the user first enters a bed with the intention of going to sleep (e.g., not if the user intends to first watch television or play with a smart phone before going to sleep, etc.), and ends on a second date (e.g., Tuesday, Jan. 7, 2020) at a second time (e.g., 7:00 AM), that can be referred to as the next morning, when the user first exits the bed with the intention of not going back to sleep that next morning.

In some implementations, the user can manually define the beginning of a sleep session and/or manually terminate a sleep session. For example, the user can select (e.g., by clicking or tapping) one or more user-selectable element that is displayed on the display device 172 of the user device 170 (FIG. 1) to manually initiate or terminate the sleep session.

Generally, the sleep session includes any point in time after the user 210 has laid or sat down in the bed 230 (or another area or object on which they intend to sleep), and has turned on the respiratory therapy device 122 and donned the user interface 124. The sleep session can thus include time periods (i) when the user 210 is using the CPAP system but before the user 210 attempts to fall asleep (for example when the user 210 lays in the bed 230 reading a book); (ii) when the user 210 begins trying to fall asleep but is still awake; (iii) when the user 210 is in a light sleep (also referred to as stage 1 and stage 2 of non-rapid eye movement (NREM) sleep); (iv) when the user 210 is in a deep sleep (also referred to as slow-wave sleep, SWS, or stage 3 of NREM sleep); (v) when the user 210 is in rapid eye movement (REM) sleep; (vi) when the user 210 is periodically awake between light sleep, deep sleep, or REM sleep; or (vii) when the user 210 wakes up and does not fall back asleep.

The sleep session is generally defined as ending once the user 210 removes the user interface 124, turns off the respiratory therapy device 122, and gets out of bed 230. In some implementations, the sleep session can include additional periods of time, or can be limited to only some of the above-disclosed time periods. For example, the sleep session can be defined to encompass a period of time beginning when the respiratory therapy device 122 begins supplying the pressurized air to the airway or the user 210, ending when the respiratory therapy device 122 stops supplying the pressurized air to the airway of the user 210, and including some or all of the time points in between, when the user 210 is asleep or awake.

Referring to the timeline 300 in FIG. 3, the enter bed time teed is associated with the time that the user initially enters the bed (e.g., bed 230 in FIG. 2) prior to falling asleep (e.g., when the user lies down or sits in the bed). The enter bed time teed can be identified based on a bed threshold duration to distinguish between times when the user enters the bed for sleep and when the user enters the bed for other reasons (e.g., to watch TV). For example, the bed threshold duration can be at least about 10 minutes, at least about 20 minutes, at least about 30 minutes, at least about 45 minutes, at least about 1 hour, at least about 2 hours, etc. While the enter bed time t_{bed} is described herein in reference to a bed, more generally, the enter time teed can refer to the time the user initially enters any location for sleeping (e.g., a couch, a chair, a sleeping bag, etc.).

The go-to-sleep time (GTS) is associated with the time that the user initially attempts to fall asleep after entering the bed (teed). For example, after entering the bed, the user may engage in one or more activities to wind down prior to trying to sleep (e.g., reading, watching TV, listening to music, using the user device 170, etc.). The initial sleep time (tₛₗₑₑₚ) is the time that the user initially falls asleep. For example, the initial sleep time (tₛₗₑₑₚ) can be the time that the user initially enters the first non-REM sleep stage.

The wake-up time t_{wake} is the time associated with the time when the user wakes up without going back to sleep (e.g., as opposed to the user waking up in the middle of the night and going back to sleep). The user may experience one of more unconscious microawakenings (e.g., microawakenings MA₁ and MA₂) having a short duration (e.g., 5 seconds, 10 seconds, 30 seconds, 1 minute, etc.) after initially falling asleep. In contrast to the wake-up time t_{wake}, the user goes back to sleep after each of the microawakenings MA₁ and MA₂. Similarly, the user may have one or more conscious awakenings (e.g., awakening A) after initially falling asleep (e.g., getting up to go to the bathroom, attending to children or pets, sleep walking, etc.). However, the user goes back to sleep after the awakening A. Thus, the wake-up time t_{wake} can be defined, for example, based on a wake threshold duration (e.g., the user is awake for at least 15 minutes, at least 20 minutes, at least 30 minutes, at least 1 hour, etc.).

Similarly, the rising time tᵣᵢₛₑ is associated with the time when the user exits the bed and stays out of the bed with the intent to end the sleep session (e.g., as opposed to the user getting up during the night to go to the bathroom, to attend to children or pets, sleep walking, etc.). In other words, the rising time tᵣᵢₛₑ is the time when the user last leaves the bed without returning to the bed until a next sleep session (e.g., the following evening). Thus, the rising time tᵣᵢₛₑ can be defined, for example, based on a rise threshold duration (e.g., the user has left the bed for at least 15 minutes, at least 20 minutes, at least 30 minutes, at least 1 hour, etc.). The enter bed time teed time for a second, subsequent sleep session can also be defined based on a rise threshold duration (e.g., the user has left the bed for at least 4 hours, at least 6 hours, at least 8 hours, at least 12 hours, etc.).

As described above, the user may wake up and get out of bed one more times during the night between the initial t_{bed} and the final tᵣᵢₛₑ. In some implementations, the final wake-up time t_{wake} and/or the final rising time tᵣᵢₛₑ that are identified or determined based on a predetermined threshold duration of time subsequent to an event (e.g., falling asleep or leaving the bed). Such a threshold duration can be customized for the user. For a standard user which goes to bed in the evening, then wakes up and goes out of bed in the morning any period (between the user waking up (t_{wake}) or raising up (tᵣᵢₛₑ), and the user either going to bed (t_{bed}), going to sleep (t_{GTS}) or falling asleep (tₛₗₑₑₚ) of between about 12 and about 18 hours can be used. For users that spend longer periods of time in bed, shorter threshold periods may be used (e.g., between about 8 hours and about 14 hours). The threshold period may be initially selected and/or later adjusted based on the system monitoring the user's sleep behavior.

The total time in bed (TIB) is the duration of time between the time enter bed time t_{bed} and the rising time tᵣᵢₛₑ. The total sleep time (TST) is associated with the duration between the initial sleep time and the wake-up time, excluding any conscious or unconscious awakenings and/or micro-awakenings therebetween. Generally, the total sleep time (TST) will be shorter than the total time in bed (TIB) (e.g., one minute short, ten minutes shorter, one hour shorter, etc.). For example, referring to the timeline 300 of FIG. 3, the total sleep time (TST) spans between the initial sleep time tₛₗₑₑₚ and the wake-up time t_{wake}, but excludes the duration of the first micro-awakening MA₁, the second micro-awakening MA₂, and the awakening A. As shown, in this example, the total sleep time (TST) is shorter than the total time in bed (TIB).

In some implementations, the total sleep time (TST) can be defined as a persistent total sleep time (PTST). In such implementations, the persistent total sleep time excludes a predetermined initial portion or period of the first non-REM stage (e.g., light sleep stage). For example, the predetermined initial portion can be between about 30 seconds and about 20 minutes, between about 1 minute and about 10 minutes, between about 3 minutes and about 5 minutes, etc. The persistent total sleep time is a measure of sustained sleep, and smooths the sleep-wake hypnogram. For example, when the user is initially falling asleep, the user may be in the first non-REM stage for a very short time (e.g., about 30 seconds), then back into the wakefulness stage for a short period (e.g., one minute), and then goes back to the first non-REM stage. In this example, the persistent total sleep time excludes the first instance (e.g., about 30 seconds) of the first non-REM stage.

In some implementations, the sleep session is defined as starting at the enter bed time (t_{bed}) and ending at the rising time (tᵣᵢₛₑ), i.e., the sleep session is defined as the total time in bed (TIB). In some implementations, a sleep session is defined as starting at the initial sleep time (tₛₗₑₑₚ) and ending at the wake-up time (t_{wake}). In some implementations, the sleep session is defined as the total sleep time (TST). In some implementations, a sleep session is defined as starting at the go-to-sleep time (t_{GTS}) and ending at the wake-up time (t_{wake}). In some implementations, a sleep session is defined as starting at the go-to-sleep time (t_{GTS}) and ending at the rising time (tᵣᵢₛₑ). In some implementations, a sleep session is defined as starting at the enter bed time (t_{bed}) and ending at the wake-up time (t_{wake}). In some implementations, a sleep session is defined as starting at the initial sleep time (tₛₗₑₑₚ) and ending at the rising time (tᵣᵢₛₑ).

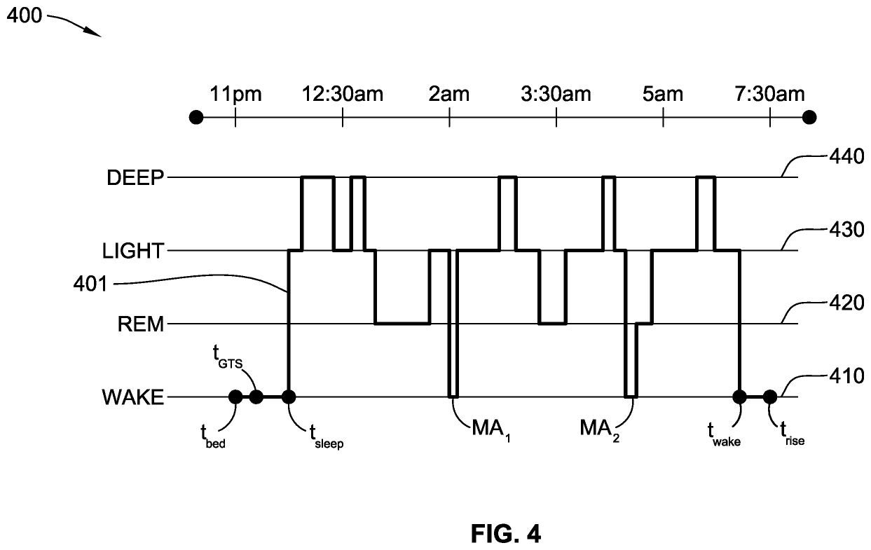

Referring to FIG. 4, an exemplary hypnogram 400 corresponding to the timeline 300 (FIG. 3), according to some implementations, is illustrated. As shown, the hypnogram 400 includes a sleep-wake signal 401, a wakefulness stage axis 410, a REM stage axis 420, a light sleep stage axis 430, and a deep sleep stage axis 440. The intersection between the sleep-wake signal 401 and one of the axes 410-440 is indicative of the sleep stage at any given time during the sleep session.

The sleep-wake signal 401 can be generated based on physiological data associated with the user (e.g., generated by one or more of the sensors 130 described herein). The sleep-wake signal can be indicative of one or more sleep states, including wakefulness, relaxed wakefulness, microawakenings, a REM stage, a first non-REM stage, a second non-REM stage, a third non-REM stage, or any combination thereof. In some implementations, one or more of the first non-REM stage, the second non-REM stage, and the third non-REM stage can be grouped together and categorized as a light sleep stage or a deep sleep stage. For example, the light sleep stage can include the first non-REM stage and the deep sleep stage can include the second non-REM stage and the third non-REM stage. While the hypnogram 400 is shown in FIG. 4 as including the light sleep stage axis 430 and the deep sleep stage axis 440, in some implementations, the hypnogram 400 can include an axis for each of the first non-REM stage, the second non-REM stage, and the third non-REM stage. In other implementations, the sleep-wake signal can also be indicative of a respiration signal, a respiration rate, an inspiration amplitude, an expiration amplitude, an inspiration-expiration ratio, a number of events per hour, a pattern of events, or any combination thereof. Information describing the sleep-wake signal can be stored in the memory device 114.

The hypnogram 400 can be used to determine one or more sleep-related parameters, such as, for example, a sleep onset latency (SOL), wake-after-sleep onset (WASO), a sleep efficiency (SE), a sleep fragmentation index, sleep blocks, or any combination thereof.

The sleep onset latency (SOL) is defined as the time between the go-to-sleep time (t_{GTS}) and the initial sleep time (tₛₗₑₑₚ). In other words, the sleep onset latency is indicative of the time that it took the user to actually fall asleep after initially attempting to fall asleep. In some implementations, the sleep onset latency is defined as a persistent sleep onset latency (PSOL). The persistent sleep onset latency differs from the sleep onset latency in that the persistent sleep onset latency is defined as the duration time between the go-to-sleep time and a predetermined amount of sustained sleep. In some implementations, the predetermined amount of sustained sleep can include, for example, at least 10 minutes of sleep within the second non-REM stage, the third non-REM stage, and/or the REM stage with no more than 2 minutes of wakefulness, the first non-REM stage, and/or movement therebetween. In other words, the persistent sleep onset latency requires up to, for example, 8 minutes of sustained sleep within the second non-REM stage, the third non-REM stage, and/or the REM stage. In other implementations, the predetermined amount of sustained sleep can include at least 10 minutes of sleep within the first non-REM stage, the second non-REM stage, the third non-REM stage, and/or the REM stage subsequent to the initial sleep time. In such implementations, the predetermined amount of sustained sleep can exclude any micro-awakenings (e.g., a ten second micro-awakening does not restart the 10-minute period).

The wake-after-sleep onset (WASO) is associated with the total duration of time that the user is awake between the initial sleep time and the wake-up time. Thus, the wake-after-sleep onset includes short and micro-awakenings during the sleep session (e.g., the micro-awakenings MA₁ and MA₂ shown in FIG. 4), whether conscious or unconscious. In some implementations, the wake-after-sleep onset (WASO) is defined as a persistent wake-after-sleep onset (PWASO) that only includes the total durations of awakenings having a predetermined length (e.g., greater than 10 seconds, greater than 30 seconds, greater than 60 seconds, greater than about 5 minutes, greater than about 10 minutes, etc.)

The sleep efficiency (SE) is determined as a ratio of the total time in bed (TIB) and the total sleep time (TST). For example, if the total time in bed is 8 hours and the total sleep time is 7.5 hours, the sleep efficiency for that sleep session is 93.75%. The sleep efficiency is indicative of the sleep hygiene of the user. For example, if the user enters the bed and spends time engaged in other activities (e.g., watching TV) before sleep, the sleep efficiency will be reduced (e.g., the user is penalized). In some implementations, the sleep efficiency (SE) can be calculated based on the total time in bed (TIB) and the total time that the user is attempting to sleep. In such implementations, the total time that the user is attempting to sleep is defined as the duration between the go-to-sleep (GTS) time and the rising time described herein. For example, if the total sleep time is 8 hours (e.g., between 11 PM and 7 AM), the go-to-sleep time is 10:45 PM, and the rising time is 7:15 AM, in such implementations, the sleep efficiency parameter is calculated as about 94%.

The fragmentation index is determined based at least in part on the number of awakenings during the sleep session. For example, if the user had two micro-awakenings (e.g., micro-awakening MA₁ and micro-awakening MA₂ shown in FIG. 4), the fragmentation index can be expressed as 2. In some implementations, the fragmentation index is scaled between a predetermined range of integers (e.g., between 0 and 10).

The sleep blocks are associated with a transition between any stage of sleep (e.g., the first non-REM stage, the second non-REM stage, the third non-REM stage, and/or the REM) and the wakefulness stage. The sleep blocks can be calculated at a resolution of, for example, 30 seconds.

In some implementations, the systems and methods described herein can include generating or analyzing a hypnogram including a sleep-wake signal to determine or identify the enter bed time (teed), the go-to-sleep time (t_{GTS}), the initial sleep time (tₛₗₑₑₚ), one or more first micro-awakenings (e.g., MA₁ and MA₂), the wake-up time (t_{wake}), the rising time (tᵣᵢₛₑ), or any combination thereof based at least in part on the sleep-wake signal of a hypnogram.

In other implementations, one or more of the sensors 130 can be used to determine or identify the enter bed time (t_{bed}), the go-to-sleep time (t_{GTS}), the initial sleep time (tₛₗₑₑₚ), one or more first micro-awakenings (e.g., MA₁ and MA₂), the wake-up time (t_{wake}), the rising time (tᵣᵢₛₑ), or any combination thereof, which in turn define the sleep session. For example, the enter bed time t_{bed} can be determined based on, for example, data generated by the motion sensor 138, the microphone 140, the camera 150, or any combination thereof. The go-to-sleep time can be determined based on, for example, data from the motion sensor 138 (e.g., data indicative of no movement by the user), data from the camera 150 (e.g., data indicative of no movement by the user and/or that the user has turned off the lights) data from the microphone 140 (e.g., data indicative of the using turning off a TV), data from the user device 170 (e.g., data indicative of the user no longer using the user device 170), data from the pressure sensor 132 and/or the flow rate sensor 134 (e.g., data indicative of the user turning on the respiratory therapy device 122, data indicative of the user donning the user interface 124, etc.), or any combination thereof.

Apnea, hyponea, subapnea and related respiratory events during a sleep session of a user often lead to blood pressure events (e.g. random spikes, continually rising blood pressure for a period of time, etc.). This exacerbates pre-existing blood pressure conditions suffered by the user, whether diagnosed or undiagnosed.

The systems and methods described herein can be configured to manage blood pressure conditions of the user of a respiratory therapy system by controlling operational parameters associated with the respiratory therapy system. This is achieved by proactively or reactively reducing or removing the occurrence of the blood pressure events and associated physiological events (e.g. sympathetic autonomous nervous system activations). The systems and methods described herein are configured to modify the operational parameters for pressurized air delivered to the user through the respiratory therapy system. This happens either intelligently in response to detecting blood pressure events and associated physiological events or in response to a recommendation from the user or a healthcare provider who is alerted upon the detection of the blood pressure events and associated physiological events.

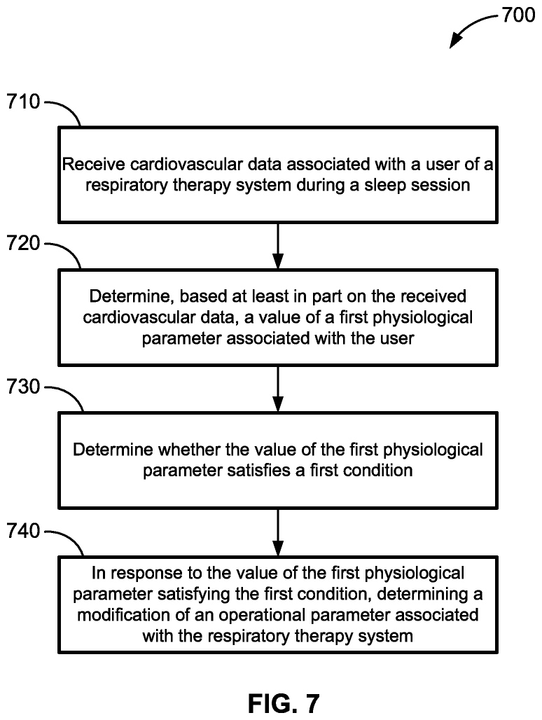

A method 700 of managing blood pressure conditions of the user 210 (FIG. 2) using the respiratory therapy system 120 is described below and illustrated with respect to a flow diagram shown in FIG. 7. Referring to the flow diagram of FIG. 7, at step 710 of method 700, cardiovascular data associated with the user 210 during a sleep session is received. The cardiovascular data is detected by one or more cardiovascular sensing mechanisms such as, but not limited to, the PPG sensor 154, an RF sensor, an infrared (IR) sensor, a pressure sensor, a displacement sensor, and the like, as described above. The cardiovascular data is related to one or more physiological parameters or signals such as, but not limited to, blood pressure, respiration rate, respiration rate variability, heart rate, heart rate variability, electroencephalogram (EEG) signal, electrocardiogram (ECG) signal, cardiac waveform, electrooculogram (EOG) signal, electromyogram (EMG) signal, pulse transit time, blood oxygenation level, and the like. The cardiovascular data is generated by a device having the one or more cardiovascular sensing mechanisms. In some embodiments, the device is the blood pressure device 180 configured to detect and generate cardiovascular data in an invasive or a non-invasive manner, as described above. The blood pressure device 180 may be positioned external to the respiratory therapy system 120, coupled directly or indirectly to the user interface 124, coupled directly or indirectly to a headgear associated with the user interface 124, or inflatably coupled to or about a portion of the user 210. Further, the blood pressure device 180 may be communicatively coupled to the respiratory therapy system 120.

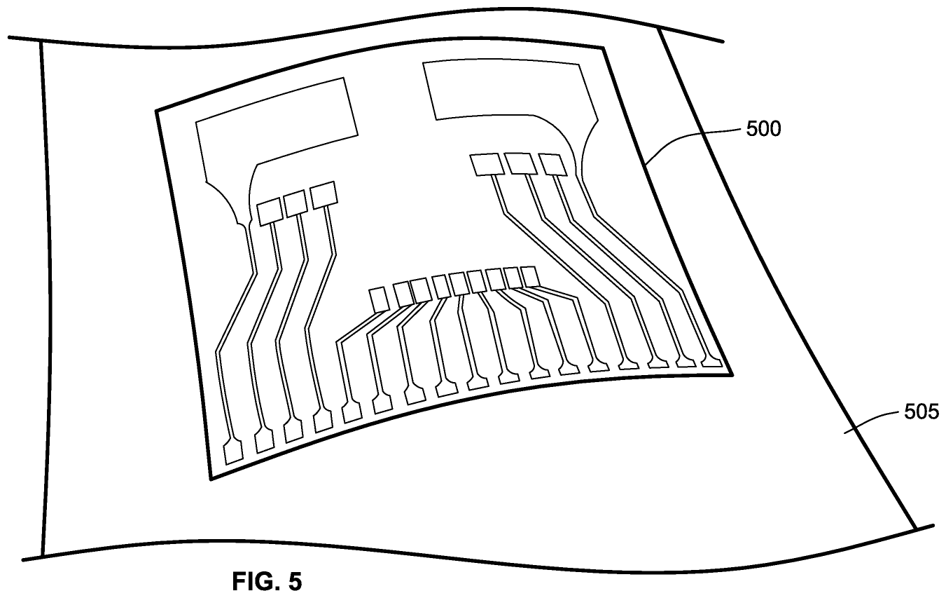

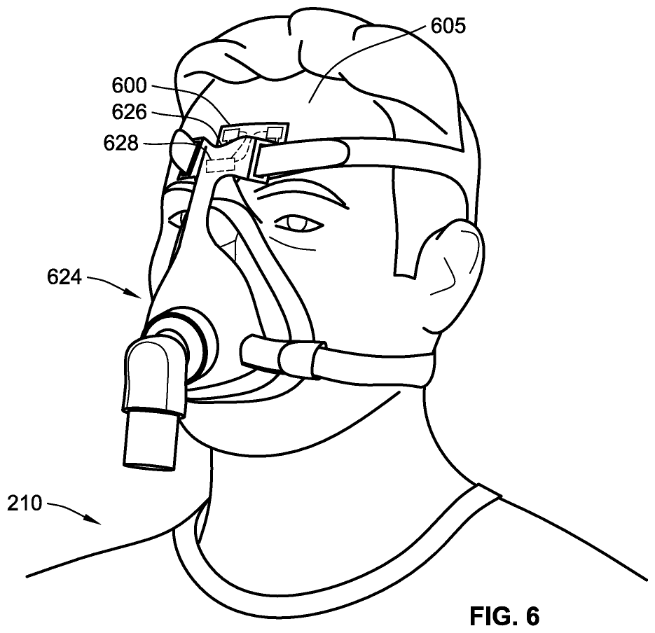

In some implementations, the device generating the cardiovascular data is a wearable smart patch 500, 600 communicatively coupled to the respiratory therapy system 120, as shown in FIGS. 5-6. FIG. 5 illustrates a smart patch 500 communicatively coupled to the respiratory therapy system 120 and disposed on a skin 505 of the user 210. FIG. 6 illustrates a smart patch 600 coupled to an inner surface 626 of the user interface 624 of the respiratory therapy system 120 and contacting a forehead skin 605 of the user 210. The smart patch 600 may be coupled to other locations (e.g. cushion, headgear) of the user interface 624 that contact the face or head of the user 210 when the user interface is worn by the user. The smart patch 600 (or any suitable form of blood pressure sensor) may be coupled to, and integrally formed with, one or more components of the user interface 624 such as the cushion, frame or headgear. In some implementations, the blood pressure sensor may be powered via wiring extending from the respiratory therapy device 122. In some embodiments, the smart patch 600 is powered by a remotely-chargeable coil 628 on the user interface 624. However, it will be understood that any suitable blood pressure device 180, communicatively coupled to the respiratory therapy system 120, may be used.

In some implementations, the device generating the cardiovascular data is a cardiovascular sensing device (not shown) communicatively coupled to the respiratory therapy system 120 and positioned remotely from the user 210 or a cardiovascular sensing device (not shown) communicatively coupled to the respiratory therapy system 120 and disposed on the user interface 124 or other component(s) of the respiratory therapy system 120.

In some implementations, the respiratory therapy device 122 may generate the cardiovascular data. The airflow parameters of the pressurized air in the respiratory therapy device 122 may be derived from flow data generated by the flow sensor 134 and/or pressure data generated by the pressure sensor 132, continuously or at predetermined time intervals. The pressure sensor 130 and the flow rate sensor 132 are typically located in the respiratory therapy device 122 in fluid communication with the pressurized air but can be coupled to or integrated in any suitable component or aspect of the respiratory therapy system 120 described herein.

At step 720, a value of a first physiological parameter associated with the user 210 is determined based at least in part on the received cardiovascular data. As noted above, the first physiological parameter may be blood pressure, respiration rate, respiration rate variability, heart rate, heart rate variability, electroencephalogram (EEG) signal, electrocardiogram (ECG) signal, cardiac waveform, electrooculogram (EOG) signal, electromyogram (EMG) signal, pulse transit time, blood oxygenation level, and the like. The value of the first physiological parameter may be a number, a range of numbers, a group of numbers, an average of numbers associated with multiple samples of the physiological parameter, an average of numbers associated with multiple sources of the received cardiovascular data, and the like.

In some implementations, the value of the first physiological parameter is a current blood pressure of the user 210. Additionally, or alternatively, the value of the first physiological parameter may be an estimated future blood pressure of the user 210. The estimated future blood pressure of the user 210 is determined based at least in part on a trend analysis of historical blood pressure data of the user 210 combined with the current blood pressure of the user 210. In some embodiments, the historical blood pressure data of the user 210 may be received from an electronic health record (EHC) communicatively coupled to the respiratory therapy system 120. In such embodiments, EHC may be received and analyzed by an external device (e.g. smartphone) or a healthcare provider. In other embodiments, the historical blood pressure data of the user 210 may be a log of values entered by a user 210 or his/her healthcare provider over a period of usage of the respiratory therapy system 120 and stored in the memory device 114 of the system 100. The estimated future blood pressure of the user 210 may also be determined based on prior analysis of variations with respect to sleep time and sleep quality metrics of the user 210, physical activity of the user 210, and indication by the user 210 regarding consumption of salt, sodium, alcohol, caffeine, beta blockers and other pharmacological substances, and the like.

In some implementations, a second value of a second physiological parameter associated with the user 210 may be determined, based at least in part on the received cardiovascular data. As a non-limiting example, if the first physiological parameter is blood pressure, the second physiological parameter may be respiration rate, respiration rate variability, heart rate, heart rate variability, electroencephalogram (EEG) signal, electrocardiogram (ECG) signal, cardiac waveform, electrooculogram (EOG) signal, electromyogram (EMG) signal, pulse transit time, blood oxygenation level, and the like. Accordingly, the second value of the second physiological parameter may be a current value or an estimated future value (which may be determined as described above in relation to an estimated future blood pressure value) of respiration rate, respiration rate variability, heart rate, heart rate variability, blood oxygenation level, etc. of the user 210.

At step 730, the method 700 determines whether the value of the first physiological parameter satisfies a first condition. The first condition may relate to a state of the first physiological parameter of the user 210. As a non-limiting example, when the value of the first physiological parameter is current blood pressure of the user 210 or the estimated future blood pressure of the user 210, then the first condition is associated with a blood pressure condition such as, but not limited to, hypotension, normal blood pressure, an elevated blood pressure, stage one hypertension, stage two hypertension, and the like. The elevated blood pressure/hypertension may occur solely, primarily or additionally while the user is sleeping and is termed nocturnal hypertension. In some embodiments, the blood pressure condition of the user 210 is customized (or otherwise normalized) for the user 210 based on the historical blood pressure data of the user 210 discussed above. In other embodiments, the blood pressure condition of the user 210 may be additionally, or alternatively, based on a population average of blood pressure for individuals having similar age, gender, body mass index (BMI), severity of diagnosed obstructive sleep apnea (OSA), and/or history of residual apnea-hypopnea index (AHI) as the user 210.

In step 730, the first condition may be satisfied when the value of the first physiological parameter exceeds a threshold value, does not exceed the threshold value, is outside a predetermined range of values, is inside the predetermined range of values, and varies, by a predetermined percentage, from the value of the first physiological parameter measured when the user 210 is not asleep or not in the sleep session. As a non-limiting example, when the first condition is associated with a blood pressure condition described above, the threshold value and/or the predetermined range of values are associated with corresponding values of the blood pressure condition.

The threshold value and/or the predetermined range of values denoting the first condition for the first physiological parameter may be calibrated prior to the sleep session or during a first portion of the sleep portion (e.g. when the user is not yet asleep). The calibration process may involve determining a periodic trend of the received cardiovascular data and validating the periodic trend against cardiovascular data obtained from one or more external sources, such as an ambulatory blood pressure monitor and/or the historical blood pressure data of the user 210 discussed above. The calibration process may be updated regularly with new data on a daily or nightly basis. As a non-limiting example, when the first physiological parameter is blood pressure, the threshold value and/or predetermined range of values for a stage one hypertension may be calibrated by determining a periodic trend of the blood pressure data received from the blood pressure device 180 and then validating the periodic trend against blood pressure data obtained separately from an external source such as an ambulatory blood pressure monitor or the historical blood pressure data of the user 210 discussed above. The calibration process can additionally take into account a variety of factors such as, but not limited to, difference between blood pressure measurements of the user 210 taken during the day and before or during an initial portion of the sleep portion, sleep quality metrics of the user 210, physical activity of the user 210, indication by the user 210 regarding consumption of salt, sodium, alcohol, caffeine, beta blockers and other pharmacological substances, and the like.

In step 730, the satisfaction of the first condition may indicate and correlate with occurrence of one or more respiratory events during at least a portion of the sleep session (e.g., at least 10% of the sleep session, at least 50% of the sleep session, 75% of the sleep session, at least 90% of the sleep session, etc.). The respiratory therapy system 120 may comprise a pressure ramping feature (e.g. an intelligent pressure ramping feature) that starts with applying a low pressure of delivered air to the user 210 and gradually increases the pressure of the delivered air, until the system detects that the user 210 has entered the sleep state (i.e. has fallen asleep). Within a period of time (e.g. about three minutes) of detecting that the user 210 has fallen asleep, the respiratory therapy system 120 automatically ramps up the pressure of the delivered air, preferably at a slow, comfortable rate, to the prescribed level for the user 210, within about thirty minutes of entering the sleep session. The pressure ramping feature typically detects that the user 210 has entered a sleep session by determining any one of (i) thirty breaths of stable breathing (roughly three minutes), (ii) five consecutive snore breaths, or (iii) three obstructive apneas or hypopneas within two minutes.

The satisfaction of the first condition may indicate that a cardiovascular event(s), such as blood pressure spikes, correlate with the occurrence of the one or more respiratory events. The one or more respiratory events may be one or more of a central apnea, an obstructive apnea, a mixed apnea, a hypopnea, a subapnea, snoring, choking, wheezing, coughing, and the like. The satisfaction of the first condition may be manifested by spikes (e.g. intermittent elevations relative to normal and/or daytime values) in values of the first physiological parameter (e.g. blood pressure), or characterized by a value continuously being over a threshold value and/or predetermined range of values, or a continuously rising value of the physiological parameter for a period of time during the sleep session, after which the value falls below the threshold value and/or predetermined range of values denoting the condition for the first physiological parameter. Any other physiological events associated with the satisfaction of the first condition may also indicate the occurrence of the respiratory event. As a non-limiting example, when the first physiological parameter is blood pressure, spikes in blood pressure readings above the threshold value and/or predetermined range of values for a blood pressure condition of the user 210 (e.g. stage one hypertension), and any associated reaction from the autonomous nervous system, may indicate that the user 210 is experiencing an apnea event, e.g. an obstructive apnea or hypopnea event.

In embodiments where a second value of the second physiological parameter associated with the user 210 is determined, the method 700 may determine whether the second value of the second physiological parameter satisfies a second condition that reduces a likelihood that the satisfying of the first condition is caused by an event unrelated to the occurrence of the respiratory event. The second condition may be satisfied when the second value of the second physiological parameter exceeds a second threshold value, does not exceed the second threshold value, is outside a second predetermined range of values, is inside the second predetermined range of values, or varies, by a second predetermined percentage, from the second value of the second physiological parameter measured when the user 210 is not asleep. The second threshold value and/or the second predetermined range of values denoting the second condition for the second physiological parameter may be calibrated prior to the sleep session or during a first portion of the sleep session (e.g. when the user is not yet asleep), using a similar calibration process described above. It is acknowledged that the occurrence of the event unrelated to the respiratory event may, nonetheless, coincide with a respiratory event and sometimes even contribute to the satisfaction of the first condition. In some embodiments, satisfying the second condition could be indicative of occurrence of mild or partial apneas or RERAs, which would not individually satisfy the first condition, but may do so by the combination of such occurrences.

As a non-limiting example, if the second physiological parameter is blood oxygenation level, then the value of the blood oxygenation level of the user 210 (such as detected by PPG sensor 154 as described herein) falling below a threshold value or outside a predetermined range of values considered appropriate blood oxygenation level for the user 210 would reduce the likelihood that satisfying the first condition for the first physiological parameter, i.e. the spikes in blood pressure readings obtained from the blood pressure device 180 for the user 210 is caused by movements of the user 210, removal of the user interface 124, partial or full awakening of the user 210, changing sleep stages of the user 210, and the like. That is, the value of the blood oxygenation level of the user 210 falling below a threshold value appropriate for the user 210 would confirm that the spikes in the blood pressure readings for the user 210 are, in fact, caused by a respiratory event such as an obstructive apnea, hypopnea, etc. In other non-limiting examples, the second physiological parameter may be a respiration rate, which may be indicative a respiratory event such as an obstructive apnea, hypopnea, etc.

At step 740, in response to the value of the first physiological parameter satisfying the first condition, a modification of an operational parameter associated with the respiratory therapy system 120 is determined. In some implementations, the operational parameter associated with the respiratory therapy system 120 may be a flow rate, a pressure, a temperature, and/or a humidity of pressurized air delivered to the user 210 by the respiratory therapy device 122. The operational parameter associated with the respiratory therapy system 120 may also be an electric current delivered to the respiratory therapy device 122 to control the flow rate, pressure, temperature, humidity of the pressurized air delivered to the user 210. The operational parameters associated with the respiratory therapy system 120 may have prescribed values, ranges, or a programs of values and/or ranges of one or more parameters selected based on the physiological parameter (e.g. blood pressure) and customized to the user's condition related to the physiological parameter (e.g. blood pressure condition).

As a non-limiting example, in response to the blood pressure readings exceeding a calibrated threshold value considered appropriate for the user 210 based on a diagnosed or undiagnosed blood pressure condition, a pressure or flow rate of the pressurized air delivered to the user 210 through the user interface 124 may be gradually increased such as, but not limited to, from 1 cm H₂O to about 5 cm H₂O, or from 3 cm H₂O to about 10 cm H₂O. Such an action is intended to proactively or reactively mitigate the occurrence of the respiratory event such as obstructive apnea, hypopnea, etc. that causes the blood pressure readings to exceed the calibrated threshold value.

The modification of the operational parameter associated with the respiratory therapy system 120 may also be based at least in part on one or more health characteristics of the user 210. As non-limiting examples, the one or more health characteristics of the user 210 may include historical cardiovascular data of the user 210, sleep quality metrics of the user 210, physical activity of the user 210, indication by the user 210 regarding consumption of salt, sodium, alcohol, caffeine, beta blockers and other pharmacological substances, and the like.

In some embodiments, the system 100 may send an alert to the user 210 and/or the user's healthcare provider indicating that the first condition has been satisfied in step 730. The alert may include a value and/or a classification of the first physiological parameter that satisfied the first condition in step 730. The alert may be an audio alert and/or a text alert sent to a smart watch, a smart phone, an activity tracker, a tablet, a smart speaker, a computer, a laptop, a server, the cloud, and the like. As a non-limiting example for when the first physiological parameter is blood pressure, an alert may be sent to the user and/or the healthcare provider that the current blood pressure has exceeded a threshold value or a predetermined range of values considered appropriate for the user 210 and include the value of the current blood pressure of the user 210. In some embodiments, the alert may also classify the first condition as detection of a suspected or undiagnosed nocturnal hypertension, for example, if the value of the current blood pressure exceeds a measured daytime value/range of values of blood pressure of the user 210 by a predetermined amount, e.g. by 20 mm Hg.

In some implementations, the operational parameter associated with the respiratory therapy system 120 (e.g. flow rate of pressurized air) may be modified automatically by the control system 110 in response to the value of the first physiological parameter (e.g. current blood pressure) satisfying the first condition. A machine-learning model used by the control system 110 may be trained (e.g., using supervised or unsupervised learning) to modify the operational parameter associated with the respiratory therapy system 120 based on comprehensive data on the values of the first physiological parameter (e.g. current blood pressure) collected from historical data of the user 210, and optionally population average values of the physiological parameter of individuals having similar age, gender, body mass index (BMI), severity of diagnosed obstructive sleep apnea (OSA), history of residual apnea-hypopnea index (AHI), or other physiological conditions as the user 210.

In some implementations, the operational parameter associated with the respiratory therapy system 120 (e.g. flow rate of pressurized air) may be modified only upon receiving an input authorized by the user or the healthcare provider in response to the alert. In some embodiments, in addition to the input, the healthcare provider may also prescribe medication, guided respiration, or breathing exercises (e.g. deep breathing) while using the respiratory therapy system 120 in order to mitigate the occurrence of cardiovascular events (e.g. blood pressure spikes) and/or respiratory events during current and future sleep sessions.

The systems and methods described herein can be advantageously used to manage blood pressure conditions of the user of the respiratory therapy system by controlling operational parameters associated with the respiratory therapy system in real time. Accordingly, unwanted blood pressure changes as well as other physiological changes related to apneas, hypopneas, etc. are proactively or reactively mitigated. The systems and method described herein enables daytime and nocturnal blood pressure controlled to e.g., <130/80 mm Hg as a universal blood pressure goal, as uncontrolled blood pressure conditions pose an increased risk of cardiovascular diseases.

Further, any trends towards nocturnal hypertension or other cardiovascular condition of the user may be improved over time. The systems and methods may be used in concert with one or more of (i) medication to manage blood pressure and other cardiovascular conditions, (ii) recommended breathing exercises to help fall asleep or decrease blood pressure prior to falling asleep, and (iii) cognitive behavioral therapy for insomnia (CBTI) to improve sleep quality metrics. In some implementations of the systems and methods described herein, medication prescribed to treat a user's respiratory or cardiac condition, e.g. blood pressure condition, may be adjusted based on the effect of the respiration therapy and/or one or more modifications of an operational parameter associated with the respiratory therapy system. Such adjustment may be user-specific (e.g., tailored to a particular user) based on collected values for the first physiological parameters. This dynamic adjustment of medication can help ensure that the user is not over- or under-medicated for his/her condition.

Accordingly, the systems and methods satisfy the dual goal of trying to avoid the “dipping” blood pressure trend (i.e., keeping blood pressure above around 110/65 mmHg at night), counterbalanced with avoiding the massive surges during REM due to apnea events (e.g., obstructive, central, and/or hypopnea events). Thus, certain aspects and features of the present disclosure provide a substantial benefit, especially where spot monitoring in the clinic or periodic cuff measures are insufficient, particularly during a sleep session.

In some implementations, cardiovascular data associated with a user of a respiratory therapy system during a sleep session, one or more values of one or more physiological parameter associated with the user determined from the cardiovascular data, and/or one or more operational parameters associated with the respiratory therapy system, such as determined via the systems and method described herein, may be used to generate a sleep performance score for the user.

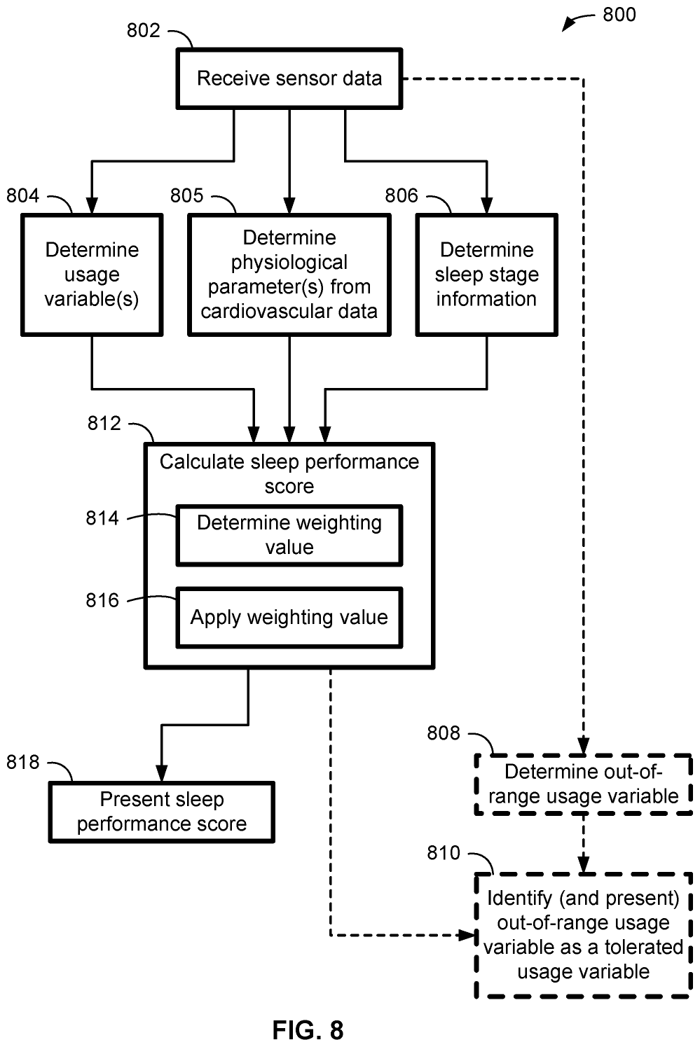

FIG. 8 is a flowchart depicting a process 800 for scoring sleep performance, according to certain aspects of the present disclosure. Process 800 can be carried out by any suitable system, such as system 100 of FIG. 1, including by processor 112 of control system 110 of FIG. 1. One, some, or all blocks of process 800 can occur during a sleep session (e.g., the given sleep session for which the sleep performance score is being calculated or a subsequent sleep session), immediately following a sleep session, or at another time. In some cases, process 800 is carried out by a user device (e.g., smartphone), such as user device 170 of FIG. 1.

At block 802, sensor data is received. The received sensor data can be collected from one or more sensors, such as one or more sensors associated with a sleep session of a user during which the user is receiving respiratory therapy from a respiratory therapy system (e.g., respiratory therapy system 120 of FIG. 1). Such one or more sensors (e.g., one or more sensors 130 of FIG. 1) can include a set of sensors of the respiratory therapy system (e.g., a pressure sensor and a flow rate sensor) and/or a set of sensors of a user device (e.g., an acoustic sensor or RF sensor of a smartphone), although other sensors can be used. In some cases, sensor data can be preprocessed prior to being received at block 802. In some cases, receiving sensor data at block 802 can include preprocessing the sensor data to improve the ability to later determine any usage variables, physiological parameter(s) from cardiovascular data, and/or sleep stage information that may be desired. In some cases, no preprocessing is performed on the sensor data.

At block 804, one or more usage variables can be determined from the sensor data. Determining one or more usage variables can include processing the sensor data (e.g., via an equation, a function, or a machine learning algorithm) to identify one or more values for the one or more usage variables. The one or more usage variables can be any number or combination of suitable usage variables, such as those disclosed herein. In some cases, a usage variable determined at block 804 can be a single-value usage variable, such as an average leak flow rate, which can be represented as a single number, or a count of detected events, which can be indicated as a single number. In some cases, however, a usage variable determined at block 804 can be a set of values, such as timestamped values, or timestamps themselves, that occur throughout the sleep session. For example, a seal quality usage variable can be represented as a collection of seal quality values (e.g., 0-100%, 0-20 on a 20-point scale, or the like) collected periodically (e.g., based on a sampling rate).

Usage variables associated with use of the respiratory therapy system can include any suitable variable related to how a user makes use of the respiratory therapy system. Examples of suitable usage variables include usage time (e.g., a duration of time the user makes use of the respiratory therapy system); a seal quality variable (e.g., an indication of the quality of seal between the user and the user interface); a leak flow rate variable (e.g., an indication of the rate of flow of unintentional leaks, such as leaks through a poor-quality seal or mouth-breathing while wearing a nasal pillow type user interface); event information (e.g., an indication of detected events that occurred during the sleep session, such as an apnea-hypopnea index (AHI)); user interface compliance information (e.g., an indication of detected user interface transition events, such as donning or removing the user interface); a number of therapy sub-sessions within the sleep sessions (e.g., a number of separate blocks of continuous usage of the respiratory therapy system); and user interface pressure. Other usage variables can be used. Statistical summaries (e.g., averages, maximums, minimums, counts, and the like) of one or more usage variables can be used as one or more additional usage variables. The one or more usage variables can include any suitable combination of usage variables.

Determining a usage variable can include processing sensor data to identify one or more values associated with the usage variable. The one or more values can be a measurement or calculated score associated with the usage variable. For example, a seal quality variable can be a measurement of leak flow rate (e.g., in L/min) or a seal quality score (e.g., 18 out of 20). Determining a usage variable can include determining a single value or multiple values (e.g., timestamped values). For example, in some cases, determining a seal quality variable can include determining a single value representative of the overall (e.g., average) seal quality throughout the sleep session (e.g., 18 out of 20). In some cases, however, determining a seal quality variable can include determining a set of timestamped values representative of the seal quality over time (e.g., on a scale of 0 to 20, 18 at 10:00:00 PM, 18.1 at 10:00:05 PM, 18.2 at 10:00:10 PM, and the like), such as data that can be charted to depict seal quality throughout a duration of time.

At block 805, one or more physiological parameters are determined from the cardiovascular data associated with the user as described herein. In some examples, the physiological parameter includes blood pressure, values for which can be generated, such as during a sleep session, as described herein.

At block 806, sleep stage information can be determined. Determining sleep stage information can include processing the sensor data to identify the sleep stage of the user at different points throughout the sleep session, such as to identify transitions between different sleep stages and durations of time spent in various sleep stages. Time spent in a sleep stage can refer to total time spent in all instances of a particular sleep stage (e.g., a total of 90 minutes of REM sleep throughout the sleep session) or time spent in individual instances of various sleep stages (e.g., a 40 minute REM stage followed by a 10 minute light sleep stage, followed by a 5 minute wakefulness stage (e.g., a microawakening), followed by a 30 minute light sleep stage, followed by a 10 minute deep stage, followed by a 15 minute light sleep stage, followed by another 20 minute REM stage). In some cases, sleep stage information can include duration of the entire sleep session. In some cases, sleep stage information can include one or more ratios between sleep stage durations and/or between each sleep stage duration and the duration of the total sleep session.

Sleep stage information can include information indicative of the sleep stages undergone by the user during the sleep session. Examples of sleep stages include a wakefulness stage, a rapid eye movement (REM) stage, a light sleep stage, and a deep sleep stage. The sensor data can be processed to determine times when the user enters and exits various stages of sleep. In some cases, determining sleep stage information can include determining a total duration of time the user spent in each sleep stage. In an example 8-hour sleep session, the sleep stage information may indicate a total of 21 minutes in wakefulness, 101 minutes in REM sleep, 267 minutes in light sleep, and 91 minutes in deep sleep. In some cases, however, determining sleep stage information can include generating timestamped data indicative of the sleep stage of the user at various times throughout the sleep session, such as data that can be charted to generate a hypnogram of the user's sleep session.

At block 812, a sleep performance score can be calculated. The sleep performance score can be calculated using the determined usage variable(s) from block 804, physiological parameter(s) determined from the cardiovascular data associated with the user from block 805, and the determined sleep stage information from block 806. In some cases, calculating the sleep performance score can include calculating one or more component scores that can be combined to calculate the final sleep performance score. In some cases, component scores can be determined for one, some, or all of the usage variables from block 804, the physiological parameter(s) from block 805, and/or the sleep stage information from block 806.

In some cases, the sleep performance score can be calculated using the determined usage variable(s) from block 804 and one or more of the physiological parameter(s) determined from the cardiovascular data associated with the user from block 805 or the determined sleep stage information from block 806. In other cases, the sleep performance score can be calculated using the physiological parameter(s) determined from the cardiovascular data associated with the user from block 805, and one of the determined usage variable(s) from block 804 or the determined sleep stage information from block 806.

In some cases, determining the sleep performance score at block 812 can include determining one or more weighting values at block 814 and applying the one or more weighting values at block 816. A weighting value can be determined for any combination of usage variables, physiological parameter(s) determined from the cardiovascular data, sleep stage information, segmented usage variables, segmented physiological parameter(s) determined from the cardiovascular data, or segmented sleep stage information. In some cases, determining weighting values can include segmenting a usage variable into multiple usage variable segments. The segments can be based on physiological parameter(s), sleep stages and/or other usage variables. For example, a usage time usage variable can be segmented based on sleep stages or an event information usage variable can be segmented based on a seal quality usage variable.

In some cases, a usage variable and/or a physiological parameter can be segmented by sleep stage, using the sleep stage information. For example, a total usage time (U) can be segmented into usage time segments using the sleep stage information, including usage time during wakefulness (U_{W}), usage time during REM sleep (U_{R}), usage time during light sleep (U_{L}), and usage time during deep sleep (U_{D}). Similar segmentation can be performed on any usage variables (e.g., seal quality segments, air leek segments, detected event segments, user interface compliance segments) or physiological parameter(s). For example, a physiological parameter that is blood pressure (P) can be segmented into blood pressure segments using the sleep stage information, including an average blood pressure during wakefulness (P_{W}), an average blood pressure during REM sleep (P_{R}), an average blood pressure during light sleep (P_{L}), and an average blood pressure during deep sleep (P_{D}). While the sleep performance score may be calculated using a number of usage variables and/or physiological parameters that are segmented, in an example with only a single physiological variable that is blood pressure, sleep performance score (Score) may be calculated according to the following equation.

Score=X_{1W}P_{W}+X_{1R}P_{R}+X_{1L}P_{L}+X_{1D}P_{D}

In this example, X_{1W} is a weighting value associated with blood pressure during wakefulness, X_{1R} is a weighting value associated with blood pressure during REM sleep, X_{1L} is a weighting value associated with blood pressure during light sleep, and X_{1D} is a weighting value associated with blood pressure during deep sleep. In some cases, the aforementioned usage variable(s), physiological parameter(s), and/or weighting values can be time-dependent.

Determining a weighting value can include accessing a pre-defined weighting value, calculating a weighting value, or receiving the weighting value (e.g., receiving the weighting value from an output of a machine learning algorithm). In some cases, the determined weighting value can be a neutral weighting value, such as a 1.0× or 100% weighting value. In some cases, the determined weighting value can be an increasing weighting value, such as a 1.5× or 150% weighting value. In some cases, the determined weighting value can be a decreasing weighting value, such as a 0.5× or 50% weighting value.

In some cases, a weighting value for a usage variable can be determined based on the sleep stage information from block 806, one or more of the physiological parameter(s) determined from the cardiovascular data associated with the user from block 805, and/or other usage variable(s) from block 804. In some cases, determining weighting values at block 814 can include determining a set of weighting values for the given usage variable and/or physiological parameter(s), such as a weighting value for each combination of the given usage variable, a given physiological parameter, and the sleep stages from the sleep stage information and/or the other usage variables. In an example, weighting values determined for an event information usage variable (e.g., detected apnea or hypopnea events) can include determining 1) a weighting value for the event information usage variable in combination with a wakefulness sleep stage; 2) a weighting value for the event information usage variable in combination with a light sleep stage; 3) a weighting value for the event information usage variable in combination with a deep sleep stage; and 4) a weighting value for the event information usage variable in combination with an REM sleep stage.

In some cases, determining a weighting value at block 814 can include applying another usage function (e.g., time-dependent usage variable) and/or physiological parameter function (e.g., time-dependent physiological parameter) to a function. For example, a weighting value for a given usage variable can be a proportional or inverse proportional function of another usage variable and/or physiological parameter.

In some cases, determining a weighting value can include accessing a database of weighting values. In some cases, accessing a database of weighting values can include using information associated with the user (e.g., physiological information and/or demographic information) to select one or more weighting values from the database of weighting values. For example, information associated with the user can be used to determine a population into which the user falls (e.g., based on an age range, gender information, geolocation, or the like) and then select one or more weighting values associated with the determined population. In some cases, health information (e.g., professional diagnoses, self-reported diagnoses, and/or health-related measurements) can be used to determine one or more weighting values.

Applying weighting values at block 816 can include applying one or more weighting values to one or more usage variables, one or more of the physiological parameter(s) determined from the cardiovascular data associated with the user, and/or sleep stage information. Applying a weighting value can include using the weighting value to calculate a component score for the usage variable and/or physiological parameter(s), and/or to calculate a sub-component score for a segmented usage variable and/or segmented physiological parameter(s). In some cases, applying a weighting value can include multiplying the weighting value by the usage variable (or segmented usage variable, physiological parameter(s), segmented physiological parameter(s), or other such value). In some cases, applying weighting values at block 816 can include applying multiple weighting values to a given usage variable, usage variable segment, physiological parameter, and/or physiological parameter segment. For example, a usage variable segment that is a usage time segment during REM sleep can have a first weighting value applied that is a weighting value calculated and/or selected specifically for usage time segments during REM sleep, as well as a second weighting value applied that is a weighting value calculated and/or selected globally for the usage variable and/or the sleep stage. For example, the first weighting value can be based on a preset weighting value and the second weighting value can be based on user information.

In some cases, calculating a sleep performance score at block 812 can be performed in other fashions while making use of the determined usage variable(s) from block 804, the one or more of the physiological parameter(s) determined from the cardiovascular data associated with the user from block 805, and/or the sleep stage information from block 806.

At block 818, the sleep performance score can be presented, such as to the user of the respiratory therapy system, a caregiver, or another entity. Presenting the sleep performance score can include presenting the sleep performance score in an easily digestible manner, such as a number (e.g., a number from 0 to 100), a percentage (e.g., a percentage from 0% to 100%), a color coded indicator, a graphical indicator (e.g., a bar or circular gauge filled according to the sleep performance score), or other such manner.

In some cases, presenting the sleep performance score at block 818 can further include presenting additional information, such as by default and/or upon receiving a trigger action (e.g., pressing of a button). In some cases, the additional information can include one or more component scores or sub-component scores. In some cases, the additional information can include a number and/or type of physiological parameters, such as blood pressure spikes during the sleep session or a portion thereof. In some cases, the additional information can include a hypnogram of the sleep stage information. In some cases, the additional information can include a summary of sleep stage information and/or a summary of one or more component scores or sub-component scores. In some cases, the additional information can include an indication of how much a component score or sub-component score contributed to the sleep performance score. In some cases, the additional information can include a recommendation for making an adjustment to the respiratory therapy system for improving the sleep performance score. For example, the recommendation can include an instruction to replace a user interface or adjust a setting on the respiratory therapy device. In some cases, the additional information can include trend data indicating a trend in sleep performance score for the given sleep session and a number of preceding sleep sessions.

In some optional cases, an out-of-range usage variable can be determined at block 808. Determining an out-of-range usage variable can be based on the sensor data received from block 802. Determining an out-of-range usage variable can be separate from and/or part of determining usage variable(s) at block 804, and can include identifying that a value of the given usage variable is out of a threshold range (e.g., below a threshold level, above a threshold level, and/or between a lower threshold level and an upper threshold level).

At optional block 810, an out-of-range usage variable can be identified as a tolerated usage variable based on the calculated sleep performance score from block 612 and the out-of-range usage variable determined from block 808. The out-of-range usage variable can be identified as a tolerated usage variable when the sleep performance score is nevertheless above a threshold value. Thus, despite the given usage variable being out of a desired range, the sleep performance score still indicates a good sleep session with use of respiratory therapy (e.g., a sleep session with high quality and/or a sleep session with efficient and/or effective use of respiratory therapy). In some cases, identifying an out-of-range usage variable as a tolerated usage variable at block 810 can further include presenting the out-of-range usage variable as a tolerated usage variable (e.g., presenting an indication that a given usage variable is well-tolerated).

In some cases, once a usage variable is identified as a tolerated usage variable, future instances of determining weighting values at block 814 can include determining an adjusted weighting value for any usage variable identified as a tolerated usage variable. The adjusted weighting value can de-emphasize the effect of the tolerated usage variable on the sleep performance score. For example, if a user well-tolerates decreases in seal quality, calculation of future sleep performance scores can apply lower weighting values to the seal quality variable.

In some cases, instead of or in addition to an out-of-range usage variable associated with block 804, an out-of-range physiological parameter can be determined and/or leveraged in association with block 805 in a similar fashion.

The blocks of process 800 can be performed in any suitable order, including certain blocks being performed simultaneously. For example, calculating sleep performance score at block 812 can occur simultaneously to determining an out-of-range usage variable. In another example, determining sleep stage information can occur after determining usage variable(s). Additionally, while process 800 is described with certain blocks, one, some, or all of the blocks of process 800 can be removed and/or replaced with other blocks. Additionally, in some cases, process 800 can include additional blocks not depicted in FIG. 8.

The systems and methods described herein removes any discomfort caused by blood pressure conditions which can cause sudden arousal and associated insomnia and lead to reduction in use and compliance of respiratory therapy provided by the respiratory therapy system. A user of the respiratory therapy system may be able to view, on a graphical user interface (e.g. graphical user interface of the display device 128), of an improvement in his/her cardiovascular conditions (e.g. a comparison of the measured blood pressure and an expected blood pressure) based on his medical conditions or lack thereof. This will enable the user to see the benefits of using the respiratory therapy system and encourage further use of the system.

One or more elements or aspects or steps, or any portion(s) thereof, from one or more of any of claims 1 to 101 below can be combined with one or more elements or aspects or steps, or any portion(s) thereof, from one or more of any of the other claims 1 to 101 or combinations thereof, to form one or more additional implementations and/or claims of the present disclosure.

While the present disclosure has been described with reference to one or more particular embodiments or implementations, those skilled in the art will recognize that many changes may be made thereto without departing from the spirit and scope of the present disclosure. Each of these implementations and obvious variations thereof is contemplated as falling within the spirit and scope of the present disclosure. It is also contemplated that additional implementations according to aspects of the present disclosure may combine any number of features from any of the implementations described herein.

1. A method comprising:
receiving cardiovascular data associated with a user of a respiratory therapy system during a sleep session;
determining, based at least in part on the received cardiovascular data, a value of a first physiological parameter associated with the user;
determining whether the value of the first physiological parameter satisfies a first condition; and
in response to the value of the first physiological parameter satisfying the first condition, determining a modification of an operational parameter associated with the respiratory therapy system.

2-6. (canceled)

7. The method of claim 1, wherein the first physiological parameter is blood pressure, respiration rate, respiration rate variability, heart rate, heart rate variability, electroencephalogram (EEG) signal, electrocardiogram (ECG) signal, cardiac waveform, electrooculogram (EOG) signal, electromyogram (EMG) signal, pulse transit time, blood oxygenation level, or any combination thereof.

8-12. (canceled)

13. The method of claim 1, wherein the satisfying the first condition includes the value of the first physiological parameter (i) exceeding a threshold value, (ii) not exceeding the threshold value, (iii) being outside a predetermined range of values, (iv) being inside a predetermined range of values, or (v) varying from the value of the first physiological parameter measured when the user is not in the sleep session by a predetermined percentage.

14. The method of claim 13, wherein (i) the threshold value, (ii) the predetermined range of values, or (iii) both (i) and (ii) are associated with a blood pressure condition, and wherein the blood pressure condition is normal blood pressure, an elevated blood pressure, stage one hypertension, stage two hypertension, or any combination thereof.

15. (canceled)

16. The method of claim 13, wherein (i) the threshold value, (ii) the predetermined range of values, or (iii) both (i) and (ii) are associated with a blood pressure condition, and wherein the blood pressure condition is (i) customized for the user based on historical blood pressure data of the user, (ii) based on blood pressure data for a population having similar age, gender, body mass index (BMI), severity of diagnosed obstructive sleep apnea (OSA), and/or apnea-hypopnea index (AHI) as the user, or (iii) a combination of (i) and (ii).

17. The method of claim 13, further comprising calibrating, prior to the sleep session or during a first portion of the sleep session, (i) the threshold value, (ii) the predetermined range of values, or (iii) both (i) and (ii), wherein the calibrating comprises:
determining a periodic trend of the received cardiovascular data; and
validating the periodic trend of the received cardiovascular data against cardiovascular data obtained from one or more external sources.

18-19. (canceled)

20. The method of claim 1, wherein the satisfying of the first condition is characterized by a continuously rising value of the first physiological parameter for a period of time during the sleep session.

21-22. (canceled)

23. The method of claim 21, wherein the satisfying of the first condition is indicative of an occurrence of a respirator event during the sleep session the method further comprising:
determining, based at least in part on the received cardiovascular data, a second value of a second physiological parameter associated with the user; and
determining whether the second value of the second physiological parameter satisfies a second condition to reduce a likelihood that the satisfying of the first condition is caused by an event unrelated to the occurrence of the respiratory event.

24. (canceled)

25. The method of claim 23, wherein the event unrelated to the occurrence of the respiratory event includes (i) movements of the user, (ii) removal of the user interface, (iii) partial or full awakening of the user, (iv) changing sleep stages of the user, or (v) any combination thereof.

26. The method of claim 1, further comprising:
in response to the value of the first physiological parameter satisfying the first condition, causing an alert indicating satisfaction of the first condition to be sent to (i) the user, (ii) a healthcare provider of the user, or (iii) both (i) and (ii).

27-30. (canceled)

31. A computer program product embodied on a non-transitory computer readable medium comprising instructions which, when executed by a computer, cause the computer to carry out the method of claim 1.

32. (canceled)

33. A system comprising:
an electronic interface configured to receive cardiovascular data associated with a sleep session of a user;
a cardiovascular sensing mechanism communicatively coupled to the electronic interface;
a control system including one or more processors configured to execute the machine-readable instructions to:
receive, from the cardiovascular sensing mechanism, cardiovascular data associated with the user during the sleep session;
determine, based at least in part on the received cardiovascular data, a value of a first physiological parameter associated with the user;
determine whether the value of the first physiological parameter satisfies a first condition; and
in response to the value of the first physiological parameter satisfying the first condition, determine modification of an operational parameter associated with the respiratory therapy system.

34-38. (canceled)

39. The system of claim 33, wherein the first physiological parameter is blood pressure, respiration rate, respiration rate variability, heart rate, heart rate variability, electroencephalogram (EEG) signal, electrocardiogram (ECG) signal, cardiac waveform, electrooculogram (EOG) signal, electromyogram (EMG) signal, pulse transit time, blood oxygenation level, or any combination thereof.

40-44. (canceled)

45. The system of claim 33, wherein the satisfying the first condition includes the value of the first physiological parameter (i) exceeding a threshold value, (ii) not exceeding the threshold value, (iii) being outside a predetermined range of values, (iv) being inside a predetermined range of values, or (v) varying from the value of the first physiological parameter measured when the user is not in the sleep session by a predetermined percentage.

46. The system of claim 45, wherein (i) the threshold value, (ii) the predetermined range of values, or (iii) both (i) and (ii) are associated with a blood pressure condition, and wherein the blood pressure condition is normal blood pressure, an elevated blood pressure, stage one hypertension, stage two hypertension, or any combination thereof.

47. (canceled)

48. The system of claim 45, wherein (i) the threshold value, (ii) the predetermined range of values, or (iii) both (i) and (ii) are associated with a blood pressure condition, and wherein the blood pressure condition is (i) customized for the user based on historical blood pressure data of the user, (ii) based on blood pressure data for a population having similar age, gender, body mass index (BMI), severity of diagnosed obstructive sleep apnea (OSA), and/or apnea-hypopnea index (AHI) as the user, or (iii) a combination of (i) and (ii).

49. (canceled)

50. The system of claim 45, wherein the control system is further configured to execute the machine-readable instructions to calibrate (i) the threshold value, (ii) the predetermined range of values, or (iii) both, by:
determining a periodic trend of the received cardiovascular data; and
validating the periodic trend of the received cardiovascular data against cardiovascular data obtained from one or more external sources.

51. (canceled)

52. The system of claim 33, wherein the satisfying of the first condition is characterized by a continuously rising value of the first physiological parameter for a period of time during the sleep session.

53. The system of claim 33, wherein the satisfying of the first condition is indicative of an occurrence of a respiratory event during the sleep session.

54. (canceled)

55. The system of claim 33, wherein the satisfying of the first condition is indicative of an occurrence of a respirator event during the sleep session, wherein the control system is further configured to execute the machine-readable instructions to:
determine, based at least in part on the received cardiovascular data, a second value of a second physiological parameter associated with the user; and
determine whether the second value of the second physiological parameter satisfies a second condition to reduce a likelihood that the satisfying of the first condition is caused by an event unrelated to the occurrence of the respiratory event.

56-101. (canceled)
